SEQUENCE LISTING (1 sequence)

SEQ ID NO: 1 - DNA, 24 bp, Artificial Sequence
  Synthetic Oligonucleotide
tcgtcgttttgtcgttttgtcgtt

(19) United States
(12) Patent Application Publication
(10) Pub. No.: US 2003/0148316 A1
(43) Pub. Date: Aug. 7, 2003

(54) Methods and compositions relating to plasmacytoid dendritic cells

(76) Inventors: Grayson B. Lipford, Dusseldorf (DE); Hermann Wagner, Eching, DE (US); Martin Zenke, Schoenow, DE (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/212,133

(22) Filed: Aug. 1, 2002

Related U.S. Application Data
(60) Provisional application No. 60/309,260, filed on Aug. 1, 2001.

(51) Int. Cl. C12Q 1/68; G01N 33/567; C12N 5/08

(52) U.S. Cl. 435/6; 435/7.21; 435/372

(57) ABSTRACT

The invention provides methods and compositions relating to a dendritic cell expression database.

### RELATED APPLICATIONS

This application claims priority to U.S. Provisional Patent Application filed Aug. 1, 2001, entitled “METHODS AND COMPOSITIONS RELATING TO PLASMACYTOID DENDRITIC CELLS”, Serial No. 60/309,260, the contents of which are incorporated by reference herein in their entirety.

### FIELD OF THE INVENTION

The invention relates to expression profiles of plasmacytoid dendritic cells either in steady state (i.e., resting state) or following treatment with immunostimulatory nucleic acids (e.g., CpG immunostimulatory nucleic acids).

### BACKGROUND OF THE INVENTION

Dendritic cells (DCs) are highly specialized antigen-presenting cells that have an essential role in the initiation and control of immune response. As “professional” antigen-presenting cells, they are function to take up, process, and present soluble antigens in complexes with either class I or class II MHC molecules (1). They are present in most tissues in a relatively immature state, but in the presence of inflammatory signals, they rapidly take up foreign antigens and undergo maturation into potent antigen-presenting cells that migrate to lymphoid organs where they initiate an immune response. Their phenotypic and functional characteristics are intimately linked to their lineage and stage of maturation. However, the expression of specific genes that mediate differentiation of pluripotent progenitors to DCs and confer lineage specific traits is largely undefined.

Dendritic cells (DCs) in their naive or so-called immature state act as environmental sentinels detecting pathogen presence and sampling interstitial fluids from which they take up and process antigen (1). Maturation of DCs to professional APCs can be initiated by T cells expressing CD40 ligand (CD40L) or directly via engagement of pathogen constituents displaying conserved molecular patterns, also termed pathogen-associated molecular patterns (PAMP) (2, 3, 4, 5, 6, 7). Maturing DCs express T cell-costimulating molecules on their surface, such as CD80, CD86, and CD40, and release soluble mediators, such as cytokines and chemokines. DCs then efficiently interact with peripheral T cells to initiate adaptive immune responses and dictate the T helper cell (Th) polarization toward either Th1 or Th2 (1).

DCs have been subdivided into lineage subsets based on surface marker phenotype. Functional characterization of human DCs has established myeloid-like DCs as Th1-inducing precursor DC type 1 (pDC1) and lymphoid-like DCs as Th2-inducing precursor DC type 2 (pDC2) (8).

pDC1 are generated from peripheral blood monocytes by treatment with GM-CSF and IL-4 and are also known as monocyte-derived DCs (MDDCs). These DCs express CD11c, CD13, CD33, and GM-CSFR (CD116), but not CD4, and become mature after stimulation with CD40L or PAMP. pDC1 production of IL-12 upon stimulation is a likely explanation for Th1 polarization.

pDC2 are plasmacytoid cells isolated from the tonsil, termed here plasmacytoid precursor DC (ppDC) (8). These cells are CD4⁺ CD11c-CD13⁻, CD33⁻, CD45RA⁺, IL-3R⁺ (CD123⁺) and use IL-3 as a survival factor (9-11). DCs of this phenotype can also be found circulating in the peripheral blood or resident in lymphoid organs (9,10, 12-16). CD4⁺/CD11⁻ DCs from the blood have also been termed plasmacytoid cells, IFN-producing cells, natural IFN producing cells, IL-3R^{high} DCs, or pDC2 (8-10, 17, 18). CD40 ligation matures ppDC, but does not induce IL-12; however, they do produce type I IFNs if stimulated with UV-irradiated HSV (8, 18). Type I IFNs (IFN-α and IFN-β) are involved in antiviral defense, cell growth regulation, immune activation, and Th1 polarization. ppDC have been implicated as the major source of type I IFNs after viral or bacterial stimulation (17, 19).

Viruses and bacteria activate MDDC and ppDC through engagement of pattern recognition receptors (e.g., Toll-like receptor (TLR) or dsRNA-responsive protein kinase). Well-documented PAMP are endotoxins (LPS), dsRNA, and immunostimulatory bacterial CpG-DNA sequences (CpG-DNA) (7). LPS, a prototypic PAMP, matures and induces cytokine production from murine bone marrow-derived DCs and human MDDC (2). An LPS binding and signalling complex assembles when TLR4 interacts with LPS bound to CD14, thus initiating the IL-1R/TLR receptor transduction pathway (20-22). CpG-DNA-driven activation of APCs also acts through the IL-1R/TLR-like signal transduction pathway; however, cellular uptake and translocation into early endosomes are required (23-25). It has been recently determined that CpG-DNA signals via TLR9 (26). TNF-associated factor-6 (TRAF-6) is a critical element in the IL-1R/TLR as well as CD40 signalling pathways (27). Subsequent to TNF-associated factor-6 both IκB kinase and Jun kinase are activated. Interestingly, dsRNA activation of dsRNA-responsive protein kinase also results in IκB kinase and Jun kinase activation (28). The convergence of these multiple stimuli may explain how they are all able to activate and mature DCs.

Bacteria and virus stimulate the release of IFNs from plasmacytoid cells; however, the PAMPs involved remain unidentified with the possible exception of dsRNA. Bacterial CpG-DNA was originally recognized for its ability to induce IFNs from both murine spleen cells and human peripheral blood cells. Given that bacterial stimuli activate DCs (29-31), we recently characterise the effects of CpG-DNA and other stimuli on human MDDC and ppDC (32). We describe that in contrast to LPS, bacterial CpG-DNA activates human lymphoid CD4⁺, CD11c⁻, ppDC cells to produce IFN-α and subsequently to mature into phenotypic DCs that display dendritic morphology, express high levels of costimulatory molecules, and produce cytokines. Conversely, LPS, but not CpG-DNA, activated myeloid MDDC/pDC1. Additionally, the effects of dsRNA and CD40 ligation were examined.

### SUMMARY OF THE INVENTION

The invention is based in part on the discovery of expression patterns for a plurality of genes, of both known and unknown function, in a purified population of plasmacytoid dendritic cells (i.e., ppDC) at both steady or resting state (i.e., untreated) and during a time course following exposure to an immunostimulatory nucleic acid. The invention provides several databases of information (see Tables provided herein) relating to these expression patterns, as well as diagnostic and therapeutic methods for employing the information in these databases. Additionally, the invention provides methods and compositions that allow simplified screening of subjects and cells according to the markers that define a plasmacytoid dendritic cell either in its resting or activated state, as defined in the databases provided herein.

In one aspect, the invention provides a method for determining a gene expression pattern in a plasmacytoid dendritic cell comprising providing an array of oligonucleotides at known locations on a solid substrate, and obtaining a hybridization pattern by hybridizing a nucleic acid expression product sample from a plasmacytoid dendritic cell to the array.

In one embodiment, the method further comprises the step of generating a database of the hybridization patterns for different pluralities of oligonucleotides. In another embodiment, the oligonucleotides are complementary to nucleic acid sequences encoding markers selected from the group consisting of cell surface markers such as signaling markers, transcription factors, growth factors, growth factor receptors, chemokines, chemokine receptors, adhesion markers, cytoskeleton markers, apoptosis regulating markers, complement regulating markers, and housekeeping markers. In another embodiment, the oligonucleotides are complementary to nucleic acid sequences of unknown function. In another embodiment, the nucleic acid expression product sample is selected from the group consisting of RNA, mRNA, cDNA, and amplified cDNA.

In one embodiment, the method further comprises determining a gene expression pattern in the plasmacytoid dendritic cell following treatment with an agent. In a related embodiment, the agent is an immunostimulatory nucleic acid molecule. In another embodiment, the immunostimulatory nucleic acid molecule is CpG immunostimulatory nucleic acid molecule.

In one aspect the invention provides a method for identifying a plasmacytoid dendritic cell comprising determining the level of expression of a PDC-specific set of markers in a test cell, and comparing the level of expression with a control, wherein a level of expression that is approximately identical to the control indicates that the test cell is a dendritic cell. As used herein, a PDC-specific marker is a marker that has been observed to be expressed by a plasmacytoid dendritic cell (pDC or PDC) according to the invention, and which was not known to be expressed prior to the present invention. In some embodiments of the invention, one PDC-specific marker is sufficient, while in others more than one PDC-specific marker is required to obtain the desired result. For example, in methods directed at isolating a pDC, it may be useful, in some instances, to use more than one marker. In methods directed at stimulating or down regulating pDC activity, it may be sufficient, in some instances, to use a single marker (and more preferably, a naturally occurring or synthetic ligand to that marker, including an antibody or antibody fragment). As used herein, the term “approximately identical” means within 20%, preferably within 10%, and even more preferably within 5% of the expression level in control.

PDC-specific markers are those markers that are listed in the tables provided herein, and include markers that are expressed by pDC in the resting state, as well as those that are induced or upregulated during immunostimulation, such as occurs with exposure to a CpG immunostimulatory nucleic acid. In important embodiments, PDC-specific markers are those that are induced or upregulated during immunostimulation; however, in some aspects of the invention, the PDC-specific markers also embrace those markers that are downregulated or completely suppressed during immunostimulation. PDC-specific markers include broad categories of markers such as cell adhesion markers (including cadherins, selectins, integrins, and CAMs), signaling molecules (including tyrosine kinases, receptor tyrosine kinases, and phosphatases), apoptosis regulating molecules, complement regulating molecules, activation molecules, costimulation molecules, chemokines and cytokines, and receptors thereof. The art is familiar with these categories as well as with species of each category.

In some embodiments, particularly those directed at the use of a single PDC-specific marker such as for example in a diagnostic or cell separation method, the invention preferably does not embrace the use of CD4, CD13, CD32, CD33, CD34, CD36, CD40, CD45RA, CD54, CD58, CD62L, CD86, HLA-DR, CD116, CD123, TNFR1 (CD120a), or CXCR3. In embodiments utilizing a PDC-marker that was heretofore not recognized as being expressed on pDC, then the use of such a marker together with any and all of the foregoing markers is provided.

In one embodiment, the PDC-specific set of markers is a set of markers expressed in an unstimulated (i.e., resting state) plasmacytoid dendritic cell, such as those markers listed in Tables 1a and 1b. In another embodiment, the PDC-specific set of markers is a set of markers expressed in a stimulated pDC, such as a pDC exposed to CpG immunostimulatory nucleic acid for 2 hours, 8 hours, or 24 hours. These markers are listed in Tables 1a, 2b and 2c, respectively.

In one embodiment, the level of expression is a level of mRNA expression. In a related embodiment, the level of mRNA expression is determined by Northern analysis, RT-PCR, or chip analysis. In another embodiment, the level of expression is a level of protein expression. The level of protein expression is determined by FACS analysis.

In one embodiment, the PDC-specific set of markers comprises at least one marker, at least two markers, at least three markers, at least four markers, at least five markers, at least ten markers, at least twenty markers, or at least thirty markers.

The PDC-specific set of markers may comprise at least one marker expressed by natural killer (NK) cells. In one embodiment, the marker expressed by natural killer (NK) cells is selected from the group consisting of NKp30, ILT2, ILT3, ILT7, LAIR1, and NK4. In another embodiment, the PDC-specific set of markers comprises a stimulatory molecule. In a related embodiment, the stimulatory molecule is selected from the group consisting of OX40 and 4-1BB ligand. In one embodiment, the PDC-specific set of markers comprises an integrin. In another embodiment, the integrin is selected from the group consisting of β7 integrins, α7 integrins, α4 integrins, β2 integrins, β3 integrins and α3 integrins (CD49). In one embodiment, the PDC-specific set of markers comprises a cell adhesion molecule. In a related embodiment, the cell adhesion molecule is selected from the group consisting of integrins, PECAM (CD31), ICAM-1 (CD54), ICAM-2 (CD102), ICAM-3 (CD50), siaoloadhesin (CD33), sialomucin (CD164), CD44, mucin (CD99) and MUC-1 (CD227). In one embodiment, the PDC-specific set of markers comprises a cytokine receptor. In another embodiment, the cytokine receptor is selected from the group consisting of IL-10 receptor, IL-1 receptor, TGF-β receptor, IL-6 receptor, IL-18 receptor and IL-17 receptor.

In another aspect, the invention provides a method of isolating plasmacytoid dendritic cells comprising isolating from a bodily sample cells that express at least one PDC-specific marker, and removing from the bodily sample cells that express a marker that is not a PDC-specific marker. In related embodiments, the cells express at least two, at least three, at least four, at least five, at least six, at least seven, at least eight, at least nine, at least ten, or more PDC specific markers.

In another embodiment, the PDC-specific marker is selected from the group consisting of 4-1BB ligand, CD40, CCR-07, CD69, CD134, IL-10R, CD83.

In one embodiment, the plasmacytoid dendritic cell is in a resting state. In another embodiment, the plasmacytoid dendritic cell has been exposed to a CpG immunostimulatory nucleic acid. The PDC-specific markers is selected from the group consisting of cell surface markers having a rank of greater than 10, greater than 25, greater than 50, greater than 75, greater than 100, greater than 125, or greater than 150 in Table 1a. In another embodiment, the PDC-specific markers is selected from the group consisting of receptors having a rank of greater than 7, greater than 10, or greater than 15 in Table 1b.

In one embodiment, the PDC-specific markers comprise at least one tyrosine kinase. In another embodiment, the PDC-specific markers comprise at least one phosphatase. In another embodiment, the PDC-specific markers comprise at least one apoptosis regulating molecule. In yet another embodiment, the PDC-specific markers comprise at least one NK cell marker. In a further embodiment, the PDC-specific markers comprise at least one co-stimulatory molecule selected from the group consisting of OX-40 and 4-1BB ligand.

In one embodiment, the bodily sample is selected from the group consisting of peripheral blood, bone marrow or lymph node tissue.

In certain embodiments, the method further comprises confirming the identity and/or maturation state of the isolated plasmacytoid dendritic cells by analyzing the level of expression of a plurality (i.e., greater than one) of PDC-specific markers in the isolated plasmacytoid dendritic cells, and comparing the level of expression to a control.

Several of the methods provided herein require comparison of a level of expression to a control. The control can be a cell known to be a pDC, or a set of data previously derived from a cell known to be a pDC. In important embodiments, the control is represented by a database of expression levels derived from resting or activated pDC. Examples of such databases include the data in the tables provided herein including the Table 1 series, Table 2 series, Table 3 series, Table 4 series, Table 5 series, Table 6 series, Table 7 series and Table 8 series.

In a further aspect, the invention provides a method for identifying a cell as a plasmacytoid dendritic cell comprising obtaining a hybridization pattern by hybridizing a nucleic acid sample from the cell to an array of oligonucleotides at known locations on a substrate (preferably a solid substrate), and comparing the hybridization pattern of the nucleic acid sample to a plasmacytoid expression database, such as those provided in Tables 1a, 1b, and the other tables contained herein. The oligonucleotides are complementary to nucleic acid sequences from markers of a plasmacytoid expression database, and a hybridization pattern of the nucleic acid pattern that is approximately identical to the plasmacytoid expression database indicates that the cell is a plasmacytoid dendritic cell.

In one embodiment, the nucleic acid sequences from a plasmacytoid expression database are selected from the group consisting of nucleic acid sequences from Tables 1a and 1b. In another embodiment, these latter nucleic acid sequences are selected from the group of sequences from Tables 1a and 1b that are cell surface markers, signaling markers and adhesion markers. In yet another embodiment, the nucleic acid sample from the cell is amplified. In a further embodiment, the nucleic acid sequences from a plasmacytoid expression database have a known function.

In one aspect, the invention provides a method for identifying an agent that modulates plasmacytoid dendritic cell activity comprising contacting a plasmacytoid dendritic cell with an agent, determining the level of expression of a PDC-specific marker, and comparing the level of expression of the PDC-specific marker to a control. An agent that effects (e.g., induces, suppresses, upregulates, or downregulates) a level of expression of a PDC-specific marker that is approximately identical to a level in the control is an agent that modulates pDC activity.

In one embodiment, the control comprises the expression level data of Tables 2a, 2b and 2c. In still other embodiments, the control comprises the expression level data of Tables 3c, 4b, 4d, 5b, 5d, 6b, 6c, 7b, 7d, 8b, and 8d. In one embodiment, the marker is selected from the plasmacytoid expression databases such as the tabled data provided herein. In one embodiment, the PDC-specific marker is a marker having a rank of greater than 20, greater than 50, greater than 100, or greater than 150 in Table 2a. In another embodiment, the PDC-specific marker is a marker having a rank of greater than 20, greater than 50, greater than 100, or greater than 150 in Table 2b. In yet another embodiment, the PDC-specific marker is a marker having a rank of greater than 20, greater than 50, greater than 100, or greater than 150 in Table 2c.

In another embodiment, the PDC-specific marker is an activation marker. The activation marker may be selected from the group consisting of OX-40 and 4-1BB ligand.

In one embodiment, the method further comprises identifying an agent that is immunostimulatory wherein the PDC-specific marker is an activation marker and the change in the level of expression is an increase in the level of expression of the marker. In another embodiment, the method further comprises identifying an agent that is immunoinhibitory wherein the PDC-specific marker is an activation marker and the change in the level of expression is a decrease in the level of expression of the marker. In yet another embodiment, the method further comprises identifying an agent that is immunostimulatory wherein the PDC-specific marker is an inhibitory marker and the change in the level of expression is a decrease in the level of expression of the marker. In yet a further embodiment, the method further comprises identifying an agent that is immunoinhibitory wherein the PDC-specific marker is an inhibitory marker and the change in the level of expression is an increase in the level of expression of the marker.

In one embodiment, the plasmacytoid dendritic cell activity is natural killer activity and the change in the level of expression of the PDC-specific marker is an increase in the level of expression of a natural killer cell activation marker. In a related embodiment, the natural killer cell activation marker is selected from the group consisting of NK4, NKp30, ILT2, ILT3, ILT7, and LAIR1, and other markers as described herein.

In still other embodiments, ppDC can be identified based on expression of novel surface markers which heretofore have not been identified as being expressed on ppDC. These novel surface markers can also be used to modulate the activity of ppDC.

In another embodiment the invention relates to the use of an agent identified using the methods of the invention for the purpose of modulating the activity of a PDC. In some embodiments these agents are mimics of CpG.

The invention provides in another aspect a method for inducing cytotoxic activity in a plasmacytoid dendritic cell comprising administering to a plasmacytoid dendritic cell an effective amount of an agent that induces cytotoxic activity such a ligand for an apoptosis regulating molecule of the databases provided herein (e.g., FasL).

In another aspect, the invention provides a method for identifying a subject responsive to treatment comprising determining the level of expression of at least 5 PDC-markers in a plasmacytoid dendritic cell population harvested from a subject and optionally exposed to CpG immunostimulatory nucleic acids, and comparing the level of expression of the at least 5 PDC-specific markers in the plasmacytoid dendritic cell population to a control. A level of expression of the at least 5 PDC-specific markers in the plasmacytoid dendritic cell population that is approximately identical (as defined herein) to the level in the control indicates that the subject is responsive to treatment. In one embodiment, the control is the data of expression levels provided in Tables 2a, 2b, or 2c.

In yet another aspect, the invention provides a method for evaluating a subject undergoing treatment (e.g., immunomodulatory treatment) comprising determining the level of expression of a PDC-specific marker in cells of the subject following administration of the treatment, and comparing the level of expression of the PDC-specific marker in the subject to a control. An level of expression of the PDC-specific marker in the subject that is approximately identical (as defined herein) to the level of expression in the control is indicative of a response to treatment in vivo.

In one embodiment, the control is a plasmacytoid expression database generated from plasmacytoid, dendritic cells exposed to CpG immunostimulatory nucleic acids. In a related embodiment, the control is data of Tables 2a, 2b or 2c. In one embodiment, the plasmacytoid expression database is generated from plasmacytoid cells administered the treatment in vitro.

In one embodiment, the method further comprises administering a second treatment to the subject. In one embodiment, the second treatment down-regulates an immune response in the subject. In another embodiment, the second treatment comprises administration of IL-10 or an antibody or antibody fragment specific for IL-10. In one embodiment, the second treatment up-regulates an immune response in the subject. In another embodiment, the second treatment comprises administration of OX-40 ligand or 4-1BB. In a related embodiment, the second treatment comprises administering a chemokine, the receptor of which is expressed according to the databases of the invention.

In another aspect, the invention provides a method of treating a subject to potentiate an immune response induced by administration of an immunostimulatory nucleic acid molecule comprising administering to a subject in need thereof an immunostimulatory agent having a receptor on the surface of a pDC in an amount effective to stimulate a pDC, wherein the agent is a ligand of the receptor or an antibody or fragment thereof specific for the receptor, and wherein the receptor on the surface of the pDC is a PDC-specific marker.

In one embodiment, the PDC-specific marker is induced following exposure to CpG immunostimulatory nucleic acids. In another embodiment, the PDC-specific marker is induced following exposure to CpG immunostimulatory nucleic acids for 2 hours, 8 hours, or 24 hours. In one embodiment, the agent is 4-1BB or OX-40 ligand. In another embodiment, the agent is a chemokine or cytokine selected from the group consisting of IL-18, IL-15, IL-6 and IL-2.

In yet another aspect, the invention provides an array of oligonucleotides consisting essentially of a planar solid support having at least a first surface, and a plurality of different oligonucleotides attached to the first surface of the solid support. Each of the different oligonucleotides is attached to the surface of the solid support at a different known location, and each of the different oligonucleotides has a different determinable sequence. The plurality of different oligonucleotides is a plurality of different markers selected from the group consisting of PDC-specific markers having a rank of greater than 5, greater than 10, greater than 15, greater than 20, greater than 30, greater than 40, greater than 50, greater than 75, greater than 100, greater than 125, greater than 150, or more (including any specific number therebetween) in the plasmacytoid expression database selected from the groups of databases consisting of Tables 1a, 1b, 2a, 2b, and 2c. The plurality of different oligonucleotides is representative of a plasmacytoid dendritic cell.

In one embodiment, the plurality of different markers is unique to a plasmacytoid dendritic cell. In another embodiment, the plurality of different markers is unique to a CpG immunostimulated pDC. In certain embodiments, each different oligonucleotide is 2-100 nucleotides in length, or 4-20 nucleotides in length. In one embodiment, each different oligonucleotide hybridizes to the a region within 1000 bases of the 3′ end of an mRNA transcript encoding a marker.

In one embodiment, the plurality of different oligonucleotides corresponds to at least 5 different PDC-specific markers. In another embodiment, the plurality of different oligonucleotides corresponds to at least 100 different PDC-specific markers. In yet another embodiment, each of the different known locations is physically separated from each of the other known locations.

In another aspect, the invention provides a solid-phase nucleic acid molecule array consisting essentially of a set of at least two nucleic acid molecules, expression products thereof, or fragments thereof, fixed to a solid substrate, wherein each nucleic acid molecule is selected from the group consisting of PDC-specific markers in the plasmacytoid expression database provided herein (e.g., Tables 1a, 1b, 2a, 2b, and 2c) and having a rank of greater than 5, great than 10, greater than 15, greater than 20, greater than 40, greater than 50, greater than 75, greater than 100, greater than 125, and greater than 150. All arrays should also include control nucleic acids that are not expressed in pDC.

In one embodiment, the solid substrate comprises a material selected from the group consisting of glass, silica, aluminosilicates, borosilicates, metal oxides, clays, nitrocellulose, or nylon. In an important embodiment, the solid substrate is glass. In preferred embodiments, each of the nucleic acid molecules are fixed to the solid substrate by covalent bonding.

In still another aspect, the invention provides a solid-phase protein microarray comprising at least two antibodies or antigen-binding fragments thereof, that specifically bind at least two different polypeptides selected from the group consisting of markers from the databases provided herein having a rank of greater than 5, great than 10, greater than 15, greater than 20, greater than 40, greater than 50, greater than 75, greater than 100, greater than 125, and greater than 150.

In another embodiment, the array further comprises at least one control polypeptide molecule. In another embodiment, the antibodies are monoclonal or polyclonal antibodies. In yet another embodiment, the antibodies are chimeric, human, or humanized antibodies. The antibodies may be single chain antibodies, but are not so limited. In another embodiment, antigen-binding fragments are F(ab′)₂, Fab, Fd, or Fv fragments.

The invention provides in one aspect, a method for a modulating plasmacytoid dendritic cell activity comprising administering to a plasmacytoid dendritic cell an immunomodulatory agent having a receptor on the surface of the plasmacytoid dendritic cell in an amount effective to modulate plasmacytoid dendritic cell activity, following exposure of the plasmacytoid dendritic cell to an immunostimulatory nucleic acid, wherein the receptor on the surface of the plasmacytoid dendritic cell is a PDC-specific marker.

In one embodiment, the immunomodulatory agent is an immunoinhibitory agent. In another embodiment, the immunomodulatory agent is an immunostimulatory agent. In one embodiment, the method further comprises modulating an immune response that is therapeutically induced by administration of an immunostimulatory nucleic acid. In another embodiment, the method further comprises modulating an immune response selected from the group consisting of a response to a microbial infection, and an autoimmune disorder.

In one embodiment, the immunomodulatory agent is at least two, at three, at least four, or at least five immunomodulatory agents. In another embodiment, the receptor on the surface of the plasmacytoid dendritic cell is a complement factor. In yet another embodiment, the complement factor is selected from the group consisting of CD55 and CD46. In one embodiment, the receptor on the surface of the plasmacytoid dendritic cell is a cell adhesion molecule. The cell adhesion molecule may be an integrin, a mucin, a selectin, or a CAM. The cell adhesion molecules may be selected from the group consisting of L-selectin (LECAM), CD164, CD44, CD43, CD87, CD47, CD81, CD162, CD147, CD11a, CD18, CD166 and CD49. In another embodiment, the receptor on the surface of the plasmacytoid dendritic cell is a cell signaling receptor. In one embodiment, the receptor on the surface of the plasmacytoid dendritic cell is a tyrosine kinase receptor. In another embodiment, the receptor on the surface of the plasmacytoid dendritic cell is a phosphatase. The phosphatase may be CD45. In another embodiment, the receptor on the surface of the plasmacytoid dendritic cell is a growth factor receptor selected from the group consisting of a cytokine receptor and a chemokine receptor. In yet another embodiment, the cytokine receptor is selected from the group consisting of IL-7 receptor (CD127), TNF receptor (CD120b), IL-4 receptor, CD132, IFN-γ receptor, IL-10 receptor, IL-1 receptor, TGFβ receptor, IL-6 receptor, IL-18 receptor, IL-17 receptor, IL-13 receptor, IL-15 receptor and IL-2 receptor. The chemokine receptor may be CD184 (CXCR4). In another embodiment, the receptor on the surface of the plasmacytoid dendritic cell is an apoptosis modulating agent. The apoptosis modulating agent may be CD95 and CD178. In one embodiment, the receptor on the surface of the plasmacytoid dendritic cell is induced following CpG immunostimulation. In another embodiment, the receptor on the surface of the plasmacytoid dendritic cell is up-regulated following CpG immunostimulation. In still another embodiment, the CpG immunostimulation is a 2 hour CpG immunostimulation or an 8 hour CpG immunostimulation. In one embodiment, the receptor on the surface of the plasmacytoid dendritic cell is expressed in an unstimulated plasmacytoid dendritic cell. In still another embodiment, the immunomodulatory agent is selected from the group consisting of an antibody or antibody fragment specific for the receptor and a ligand for the receptor. In one embodiment, the receptor on the surface of the plasmacytoid dendritic cell is ILT7, 4-1BB ligand, or OX-40.

These and other objects of the invention will be described in further detail in connection with the detailed description of the invention.

### DETAILED DESCRIPTION OF THE INVENTION

The invention is based in part on the purification and analysis of a population of plasmacytoid dendritic cells (ppDC). ppDC are now recognized as being a major cellular target of immunostimulatory agents such as immunostimulatory nucleic acids, including CpG immunostimulatory nucleic acids. These bone marrow derived cells function by scavenging the body for sites of injury, inflammation, and disease, migrating to such sites, engulfing and processing antigens from such sites, migrating to secondary lymphoid tissues (such as the lymph nodes), and presenting processed antigen to other immune cells such as T cells and B cells. The ability to harvest such cells to virtual purity enables a vast number of applications, including identification and categorization of cell types, subjects, drugs, and therapeutic regimens.

Several of the aspects of the invention relate to a compilation of data from a hybridization of transcripts (in the form of cDNA) from a purified ppDC population to a gene chip. The U95A gene chip used was provided by Affymetrix (Santa Clara, Calif.) and contains roughly 12,000 sequences, all of which have been previously characterized in terms of function or disease association. Transcript preparation, conversion to cDNA, and hybridization and readout of results from the chip are described in greater detail in the Examples, and are either routine to the ordinary artisan or are described in U.S. Pat. Nos. 6,261,776; 6,239,273; 6,197,506; 6,040,138.

The invention provides various sets of data including resting state expression data for pDC, and activated state expression data for pDC following exposure to CpG immunostimulatory nucleic acids for various periods of time (e.g., 2 hours, 8 hours, and 24 hours).

All databases provided herein have been corrected for background binding.

The data also includes expression levels of 2 hour unstimulated samples. This data was obtained by hybridizing cDNA from a sample of cells cultured for 2 hours in the absence of CpG immunostimulatory nucleic acid. The measurement is referred to herein as an absolute measurement as it has only been corrected for background binding. Because each of the nucleic acids fixed to the gene chip possess an individual background reading (i.e., presumably because each binds non-complementary control nucleic acid to differing degrees from the others), it is preferred to subtract individual backgrounds from their respective test measurements in order to arrive at the absolute measurement. The degree of hybridization to the cDNA sample to each location on the chip is measured in terms of fluorescent signal emanating from that location.

As used herein, every gene or nucleic acid fixed to the gene chip is referred to as a marker. Accordingly, the nucleic acid that binds to that fixed region is also referred to as a marker, as the sequences must be complementary for binding to occur. The function of the marker may be known, but this is not necessary. For some aspects of the invention, a marker can simply be a way of identifying (and in some cases, uniquely identifying) a cell, a patient, an immune response, etc. For some markers in the database, a more detailed description of the depository information corresponding to a particular accession number is provided.

It is well within the skill and knowledge of the ordinary artisan to determine the function that has heretofore been ascribed to each marker by searching the web sites of either GenBank or other suitable depository for the accession number, or in some instances by searching the web site of the Weismann Institute (Israel) for their “gene card” profile of accession numbers. This latter service is helpful in identifying not only all heretofore functions ascribed to a particular deposited marker, but also provides other names by which the marker has been referred, and links to medline literature that describes studies on the marker in greater detail. Accordingly, when the specification teaches that markers of a particular function can be selected, it is well within the realm of the ordinary artisan to determine which of the PDC-specific markers of the tables provided herein fall within these functional categories. It should be further noted that the functional information provided in the tables lists some markers generically as “receptors” where the marker is known to bind to another molecule. In some instances, both ligands and receptors are listed as receptors, because both partners of the binding pair are involved in a binding or interaction. The functional information also generally provides the name of the marker (as used in the prior art) and this name can be referred to in determining whether the marker is a ligand or a receptor.

The tables provided herein each represent a database of expression levels and patterns for a plurality of markers. When referred to herein, the markers of these databases are listed within the specification as if each and every accession number within the database is expressly recited herein.

Similar to the data described above, measurements of the degree of hybridization of cDNA samples deriving from cells cultured for 8 and 24 hours respectively, in the absence of CpG immunostimulatory nucleic acid, and from cells cultured for 2, 8, and 24 hours respectively in the presence of CpG immunostimulatory nucleic acids have also been determined (as described in the Examples). All measurements have had background fluorescent measurements subtracted from them.

The data provided herein include an analysis of genes that are expressed in resting (i.e., non-stimulated) pDC. These genes include cell surface markers which can be used for the isolating and identification pDC, as well as cytokine and chemokine receptors which can be exploited to stimulate resting pDC, or to further enhance a pre-existing immune response. The kinetic analysis of cell surface markers including cytokine and chemokine receptors lends insight into treatment strategies for both enhancing and suppressing an immune response that involve pDC. For example, the expression of a cytokine receptor by a resting pDC indicates that the cell will be responsive to the ligand for that receptor, and further that the cell may be activated by that ligand in the absence of other stimulants such as CpG immunostimulatory nucleic acids. Moreover, expression of a cytokine receptor following pDC stimulation (e.g., induced by exposure to CpG immunostimulatory nucleic acids) indicates that the cell is made responsive to the ligand for that receptor as a result of increased immunostimulation and that its activation state may be heightened by exposure to the ligand. In yet another example, expression of a negative regulating receptor or marker by a stimulated pDC indicates an avenue of immunoregulation of such cells where it is desired to control or suppress an inappropriate immune response. With respect to this latter embodiment, it may sometimes be desirable to control a CpG induced immune response (e.g., a clinically induced immune response), and this can be achieved by administration of an agent that binds to the negative regulatory marker on the activated pDC.

Listed herein are several categories of cell surface receptors and biologically active agents. One of ordinary skill in the art will be able to determine the appropriateness of the other markers provided herein in the methods described. While examples of cell adhesion molecules, signal transduction molecules, apoptosis regulating molecules, complement regulating molecules, and the like are provided herein, these lists are not intended to be exhaustive and one of ordinary skill in the art will be able to identify other species of each category from the tables provided herein.

The tables provided herein generally rank markers according to expression level, with those markers at the top of a list having greater expression in the cell than those markers below. The accession numbers provided represent the Genbank entry used as a template for the target sequence from which the probe-set was designed. Gene name represents the accepted name for that gene sequence, and in most instances some functional information is provided, although those of ordinary skill in the art will be able to determine the function of each marker either inherently or by simple reference to the prior art teachings. In all of the tables, markers such as MHC class I and II markers have been generally deleted due to individual specificity; however, it is to be understood that these markers are also expressed at high levels in pDC populations either in the resting or stimulated state. Accordingly, the isolation and identification strategies provided herein may include such markers. Duplicates sometimes appear in the table because more than one gene bank entry may have been used as a template for the target sequence from which the probe-set was designed.

With respect to the resting state pDC, CD123 (i.e., the IL-3 receptor, accession number D49410) is expressed. This receptor is known to be highly expressed on pDC. Both MHC II and CD123 have been used to select pDC from bulk samples. However, the majority of the remainder of markers have not heretofore been identified as present on pDC. One of the more surprising findings is the expression of natural killer (NK) cell markers by pDC. One example of such a marker is NKp30 (accession number Y14768), which is an activation receptor for induced killing. Another unexpected example is ILT3 (accession number AF072099), which is an inhibitory receptor found on NK cells. Apart from their functional virtues, these markers can be used as discriminatory markers allowing for the physical separation of pDC from other cells, and more particularly from NK cells. For example, a purification strategy for pDC can include the steps of isolating cells that express NKp30 (and optionally ILT3), followed by a step of selecting for cells that express CD68 or CD205 which are not expressed on NK cells. The method could also include rather than a positive selection step, a negative selection step. The information provided herein allows for fine-tuning of isolation and identification strategies as the vast majority of the markers listed were not heretofore recognized as being expressed by pDC.

The data also identify molecules that can be exploited to regulate (either in an upward or a downward manner) immune responses. For example, the costimulatory molecules OX40 (accession number S76792) and 4-1BB ligand (accession number U03398) are both expressed by pDC.

The following tables list in decreasing expression level markers that are expressed by pDC in a resting state.

TABLE 1a
Cell Surface Markers Expressed in Resting State pDC

| Rank | Accession # | Gene name |
|---|---|---|
| 1 | Y14768 | NKp30, NK cell receptor |
| 2 | M13560 | CD074, MHC II, invariant chain |
| 3 | AI540925 | proteoglycan, glypican 3 |
| 4 | M80244 | CD098 associated, 4F2 light chain |
| 5 | J04182 | CD107a, lysosomal, LAMP1 |
| 6 | M63438 | Ig-K constant, ig kappa chain c region |
| 7 | AF072099 | ILT3 |
| 8 | Z11697 | CD083, blast marker for DC |
| 9 | D28137 | BST-2 |
| 10 | J02939 | CD098, regulation of activation |
| 11 | M93221 | CD206 |
| 12 | X67301 | Ig-M constant |
| 13 | AF029750 | MHC, chaperone |
| 14 | X62744 | MHC-II, HLA-DMA |
| 15 | M25280 | CD062L |
| 16 | D11086 | CD-132 |
| 17 | D49410 | CD-123, IL-3RA, alpha chain |
| 18 | X14046 | CD037, 4TM B cell signaling |
| 19 | M37033 | CD053, 4TM |
| 20 | U87947 | emp-3, ymp protein |
| 21 | X81817 | Ig, signalling |
| 22 | Z49107 | galectin 9 |
| 23 | X62654 | CD063, LAMP-3 |
| 24 | Y00062 | CD045 |
| 25 | M32315 | CD120b, TNF-R2, p80 (p75) |
| 26 | D14043 | CD164 MUC-24, sialomucin |
| 27 | L05424 | CD044, homing, signaling |
| 28 | Z50022 | pituitary tumor-transforming 1 interacting |
| | | protein |
| 29 | X01060 | CD071 |
| 30 | AF043129 | CD127, IL-7RA |
| 31 | X60592 | CD040, signaling |
| 32 | J04168 | CD043 |
| 33 | U09937 | CD087 |
| 34 | U66711 | LY6E |
| 35 | M16279 | CD099, mucin |
| 36 | M12886 | TCRB, T-cell receptor, beta cluster |
| 37 | M29696 | CD127, IL-7RA |
| 38 | X96719 | lectin, C-type, AICL |
| 39 | AB006782 | galectin 9 |
| 40 | HG2147-HT2217 | mucin |
| 41 | X57809 | Ig-L, Immunoglobulin lambda locus |
| 42 | AF041261 | ILT7 |
| 43 | AF004230 | ILT2 |
| 44 | D26579 | CD156 |
| 45 | X69398 | CD047 |
| 46 | X70326 | adhesion, MacMarcks, integrin activation |
| 47 | Z22576 | CD069 |
| 48 | X74039 | CD087 |
| 49 | L06797 | CD184 |
| 50 | Y00638 | CD045 |
| 51 | X52425 | CD-124, IL-4R |
| 52 | X07979 | CD029 |
| 53 | Y00638 | CD045 |
| 54 | M33680 | CD081 |
| 55 | U25956 | CD162, psgl-1 |
| 56 | X64364 | CD147 |
| 57 | X95876 | CD183, CXCR-03 |
| 58 | Y00796 | CD011a |
| 59 | U19247 | CD119 |
| 60 | M58286 | CD120a, TNF-R1, p55 |
| 61 | M15395 | CD018, beta-2 integrin |
| 62 | M24283 | CD054, ICAM-1 |
| 63 | L05424 | CD044 |
| 64 | M14219 | decorin |
| 65 | AF013249 | LAIR1 |
| 66 | M31516 | CD055 |
| 67 | M59040 | CD044, homing, signaling |
| 68 | U40282 | integrin, ILK, signalling |
| 69 | X63717 | CD095, apoptosis |
| 70 | Y10183 | CD-166, ALCAM |
| 71 | Y00285 | CD222 |
| 72 | X94630 | CD097 |
| 73 | U91512 | ninjurin |
| 74 | D83597 | CD180 |
| 75 | U36336 | CD107b, LAMP-2 |
| 76 | M63959 | alpha-2-mrap |
| 77 | U41767 | integrin |
| 78 | D49396 | Apo-1 |
| 79 | M32334 | CD102, ICAM-2 |
| 80 | X59408 | CD046 |
| 81 | M68892 | integrin B7 |
| 82 | J02973 | CD141 |
| 83 | X16983 | CD049d |
| 84 | M38690 | CD009 |
| 85 | Y00636 | CD058, LFA-3 |
| 86 | AF098641 | CD044RC |
| 87 | M37766 | CD048, ligand CD2 |
| 88 | S71043 | CD079a |
| 89 | Y00093 | CD011c |
| 90 | HG3477-HT3670 | CD004 |
| 91 | S76792 | CD134, OX40 |
| 92 | U03398 | 4-1BB ligand |
| 93 | Z83844 | galectin-1 |
| 94 | M15059 | CD023 |
| 95 | M27533 | CD080 |
| 96 | AL022310 | OX40 ligand |
| 97 | U52112 | CD171 |
| 98 | X83490 | CD095, Apo-1 |
| 99 | U04343 | CD086 |
| 100 | X15606 | CD102, ICAM-2 |
| 101 | X72012 | CD105 |
| 102 | M59941 | CD-131 |
| 103 | L25851 | CD103 |
| 104 | N90866 | CD052 |
| 105 | M59941 | CD-131 |
| 106 | X77196 | CD107b, LAMP-2 |
| 107 | AF025533 | ILT5 |
| 108 | M23197 | CD033, sialoadhesin |
| 109 | M12807 | CD004 |
| 110 | X60708 | CD026, dipeptidyl peptidase iv |
| 111 | X69819 | CD050, ICAM-3 |
| 112 | M84349 | CD059 |
| 113 | X74328 | integrin, alpha7b |
| 114 | M73832 | CD-116 |
| 115 | L12002 | CD049d, integrin alpha 4 |
| 116 | L34657 | CD031, PECAM-1 |
| 117 | X15606 | CD102, ICAM-2 |
| 118 | U33017 | CD150, SLAM |
| 119 | X62822 | CD075 |
| 120 | AF025527 | ILT6 |
| 121 | J05582 | mucin |
| 122 | X83492 | CD095, apoptosis, Fas, Apo-1 |
| 123 | X89101 | CD095 |
| 124 | M12824 | CD008a |
| 125 | D79985 | DGCR2/IDD |
| 126 | U02687 | CD135, FLT3, STK-1 |
| 127 | M28827 | CD001c |
| 128 | AF025531 | ILT1, LIR-7 |
| 129 | U10886 | CD148 |
| 130 | L34657 | CD031, PECAM-1, diapedesis |
| 131 | L40385 | integrin |
| 132 | D84276 | CD038 |
| 133 | AF012023 | integrin |
| 134 | U37139 | signalling, integrin |
| 135 | J05581 | CD227, MUC-1, mucin 1 |
| 136 | X03066 | MHC, HLA |
| 137 | Z70519 | CD095, apoptosis |
| 138 | M58597 | CD015, ELAM-1 |
| 139 | M27492 | CD121a, IL-1R1 |
| 140 | HG371-HT26388 | mucin |
| 141 | AF011333 | CD205, DEC-205 |
| 142 | M59911 | CD049c, integrin, alpha3 |
| 143 | U34624 | CD006 |
| 144 | M54992 | CD072 |
| 145 | M64925 | red cell |
| 146 | L05424 | CD044, homing, signaling |
| 147 | U12471 | thrombospondin |
| 148 | M28170 | CD019 |
| 149 | X16863 | CD-016 |
| 150 | M16336 | CD002 |
| 151 | HG2320-HT2416 | integrin, beta3 |
| 152 | M31932 | CD032, Fc, Fc-RIIA, low affinity IgG |
| 153 | AF060231 | CD111, poliovirus receptor-like 1 |
| 154 | D30756 | OVARIAN CARCINOMA ANTIGEN CA125 |
| 155 | AC005525 | CD087 |
| 156 | D37781 | CD148 |
| 157 | M15059 | CD023, Fc, IgE |
| 158 | M98399 | CD036, scavenger receptor |
| 159 | X52228 | CD227, MUC-1, mucin 1 |
| 160 | D21878 | CD157, BST-1, adp-ribosyl cyclase 2 |
| 161 | S70348 | CD061, structural protein, integrin, beta 3 |
| 162 | U03397 | CD137, 4-1BB |
| 163 | M14648 | CD051 |
| 164 | J02931 | CD142, coagulation factor III |
| 165 | U48705 | CD167a, DDR1 |
| 166 | D38122 | CD178, FasL, APT1LG1 |

TABLE 1b
Cytokine and Chemokine Receptors Expressed in Resting State pDC

| Rank | Accession # | Gene name |
|---|---|---|
| 1 | L08177 | chemokine, EBI2, ebv-induced g protein-coupled |
| | | receptor 2 |
| 2 | U00672 | cytokine, IL-10R alpha |
| 3 | L31584 | chemokine, ccr-07, EBI-1 |
| 4 | X52015 | cytokine, IL-01RA, antagonist |
| 5 | D50683 | cytokine, TGFBR2, tgf-beta receptor type ii |
| | | precursor |
| 6 | M37435 | cytokine, M-CSF, CSF-1 |
| 7 | AF072902 | cytokine, IL-06R, gp130, signalling |
| 8 | U20350 | chemokine, CX3CR-01 |
| 9 | U43672 | cytokine, IL-18R1, Interleukin 18 receptor 1, |
| 10 | D10925 | chemokine, ccr-01, |
| 11 | U58917 | cytokine, IL-17R |
| 12 | Y10659 | cytokine, iI-13RA |
| 13 | AF014958 | chemokine, like-CCR2 |
| 14 | U41804 | cytokine, IL-01RL1LG, T1/ST2 receptor binding |
| | | protein |
| 15 | AF035279 | cytokine, iI-15RA |
| 16 | X01057 | cytokine, IL-02RA |
| 17 | U95626 | chemokine, ccr-05, mip-1-alpha, mip-1-beta |
| | | and rantes |
| 18 | U31628 | cytokine, iI-15RA |
| 19 | U68030 | chemokine, ccr-06 |

In one aspect, the invention relates to a method for isolating a ppDC based in part on cell surface markers expressed by such cells. A purification protocol may include separation on the basis of expression of CD123 (i.e., IL-3R), and preferably includes separation on the basis of at least one other, more preferably two other, and most preferably at least three markers. The markers useful in a pDC cell purification can be selected from the group consisting of markers in the databases provided in the tables, particularly Tables 1a and 1b. In important embodiment, the markers are cell surface markers. As used herein, a cell surface marker is a marker which when expressed at the polypeptide level is at the cell surface such that some portion of the polypeptide is extracellular. Additionally, DPC can be isolated via a negative selection process by depleting cells from a population that express markers that are not expressed by pDC.

A known method for isolating ppDC is to deplete lineage marker positive cells, such as CD3, CD19, CD56 and then positively select for CD123 ands MHC class II. When this technique is used plasmacytoid DC are isolated which are characterized as positive for CD4, CD11a, CD18, CD32, CD36, CD38, CD40, CD44, CD45RA, CD49d, CD54, CD58, CD62L, CD95, CD123 and MHCII (9, 10, 11). Upon microarray analysis, 151 surface markers are expressed at 2 h in the unstimulated cells. This group represents a wide array of molecules with widely divergent functions. This fingerprint will allow for more exact definition of pDC and provide agonist/antagonist targets.

pDCs have been characterized to an extent according to a subset of surface expression markers using FACS analysis. For example, Olweus et al (PNAS, 94:12551-12556, 1997) reported that pDC were positive for CD4, CD13 (weak staining), CD32, CD33, CD34 (very weak), CD36, CD40, CD45RA, CD54, CD58, CD62L, CD86 and HLA-DR. Kohrgruber et al. (J. Immunol. 163:3250-3259, 1999) reported that pDC were positive for CD116, CD123 and TNFR1 (CD120a), and negative for CD13, a finding that is inconsistent with that of Olweus et al. Cella et al. (Nat. Immunol. 1(4):305-310, 2000) reported the cell surface expression of CXCR3 on pDC. The data provided herein generally confirms these findings as most of the positive markers have been detected using the chip methods of the invention. The data of the present invention ranks these previously reported markers as follows: CD62L (rank 15); CD123 (rank 17); CD40 (rank 31); CD45 (rank 53); CD120 (rank 60); CD54 (rank 62); CD58 (rank 85); CD4 (rank 90,109); CD86 (rank 99); CD33 (rank 108); CD116 (rank 114);CD32 (rank 152); CD36 (rank 158); CD13 (rank absent); CD34 (rank absent); and CXCR-3R (rank 8 of chemokine receptor table)

Unexpectedly, several markers found to be expressed by ppDC by the microarray analysis such as, NKp30, ILT2, ILT3, ILT7 and LAIR1, are typically found on natural killer cells. ppDC also express NK4 (AA631972), and granzyme B (M17016). (See Table 1.) Although other NK marker are absent such as NKp44 (AJ010099). Such markers imply that pDC can have killing potential like cytolytic T cells or natural killer cells. Such knowledge leads to the development of clinical applications based on targeted killing by ppDC perhaps through ADCC or other mechanisms. Additionally because the ILT molecule is a negative signaling receptor, methods may be devised to inhibit this killing as well as inhibit general ppDC activation.

Thus, in one aspect the invention provides an isolated plasmacytoid dendritic cell. As used herein, an isolated plasmacytoid dendritic cell is a cell defined by the expression and/or lack of expression of one or more markers as listed in the databases of the invention and preferably those of Tables 1a and 1b, which is separated from the environment in which it normally exists, and which is readily manipulated via in vitro and ex vivo techniques. An isolated cell is one that is separated from the majority of other different cells with which it is normally in contact in vivo. In preferred embodiments, the isolated cell is also purified, meaning that the cell population in which it exists in vitro is greater than 95% pure (i.e., greater than 95% of the cells are the same as the isolated cell (e.g., a ppDC), more preferably greater than 97% pure, and most preferably greater than 99% pure. The newly mined data provided herein allows for such higher purities to be achieved.

The data provided in the databases also allow for the identification and isolation of subsets of cells within the ppDC population. For example, immature and mature ppDC can be harvested from the ppDC population either prior to in vitro stimulation, or in some preferred instances, following various times of exposure to an immunostimulatory agent. In one embodiment, a mature subset of ppDC can be isolated from the general ppDC population described here by selecting for cells that express markers having ranks of greater than 5, and in some cases, greater than 10 at the 24 hour CpG stimulation time point. Additionally, mature cells can be selected based on negative selection. Alternatively, if an immature subset is desired, it is preferable to isolate cells based on those markers expressed and not expressed at the 2 hour unstimulated time point.

In one aspect of the invention, a method is provided for identifying a plasmacytoid dendritic cell comprising determining the level of expression of at least 5 markers in a test cell, and comparing the level of expression of the at least 5 markers in the test cell with the level of expression in a plasmacytoid expression database. A level of expression of the at least 5 markers in the test cell that is approximately identical to the level of expression of that at least 5 markers in the plasmacytoid expression database indicates that the test cell is a dendritic cell. A level of expression that is approximately identical to the level of expression in the database is defined as within (i.e., +/−) 20% for measurements of individual markers, preferably within 10%, and even more preferably within 5% of the database expression level for the particular marker. As an example, an expression level of CD40 in a test cell that is +/−20% of the level of expression of CD40 in the 2 hour unstimulated data set is approximately identical to the level of the database. In this latter example, if the level of expression was for example 30 000, then this level of expression would not be considered approximately identical, but rather would be characterized as up-regulated relative to the expression level in unstimulated ppDC.

Several aspects of the invention relate to the discovery of the expression pattern within plasmacytoid dendritic cells (ppDC), and the change in that expression pattern following exposure to an immunostimulatory agent. The expression pattern provides information regarding the nucleic acid molecules that are expressed by ppDC in the steady state (i.e., unstimulated) and during a time course following exposure to an immunostimulatory agent. The steady state expression pattern (sometimes referred to herein as the 2 hour unstimulated expression level) is characteristic of the expression pattern of ppDC in vivo. As such, this expression pattern can be regarded as a nucleic acid expression “fingerprint” or “blueprint” of a ppDC. This fingerprint can be used to identify or detect a ppDC within a population of cells, to isolate a ppDC from a population of cells, to confirm the identity of an isolated ppDC, to screen a ppDC for any differences in its expression pattern relative to the fingerprint presented herein, to screen subjects for responsiveness to therapy, etc.

The data also provide information regarding the change in expression pattern following exposure to an immunostimulatory agent, such as an immunostimulatory nucleic acid molecule. In the Examples provided herein, a population of ppDC were exposed in vitro to a CpG immunostimulatory nucleic acid molecule, and at various times during this exposure (i.e., 2 hours, 8 hours, and 24 hours), cells were harvested and their expression pattern determined. Thus, the invention further provides information regarding the genes that are induced, suppressed, up-regulated, down-regulated, or unaffected by exposure to CpG immunostimulatory nucleic acid molecules. This information also allows for ppDC to be screened for their ability to respond in vivo to administration of immunostimulatory agents, such as immunostimulatory nucleic acid molecules (e.g., CpG immunostimulatory nucleic acid molecules). This latter screening in turn allows for screening of subjects who are most likely to benefit from treatment with such agents.

Stimulated sample data also provides insight into the genes that CpG immunostimulatory nucleic acids impact upon. Unlike the methods of the prior art that study the effect of a particular treatment on individual genes or nucleic acid molecules, the approach adopted herein allows for a plurality of nucleic acid molecules to be analyzed concurrently, thereby allowing one to determine the overall effect of the particular treatment on the expression of a vast number of nucleic acid molecules.

The data provided herein further allows identifies genes expressed in pDC following immunostimulation as a result of exposure to CpG immunostimulatory nucleic acids. The following tables list some of these markers.

TABLE 2a
Top 125 positively upmodulated genes by CpG-DNA (2 hours) in human pDC

| Rank | Sort score | Accession | Gene Name, function |
|---|---|---|---|
| 1 | 246.12 | X02956 | receptor, soluble, IFN-1, IFNa-05 |
| 2 | 228.66 | V00551 | receptor, soluble, IFN-1, IFNa-10 |
| 3 | 193.06 | V00535 | receptor, soluble, IFN-1, IFNb-01 |
| 4 | 189.71 | X58822 | receptor, soluble, IFN-1, IFN-omega-1 |
| 5 | 186.11 | M27318 | receptor, soluble, IFN-1, IFNa-04b |
| 6 | 185.65 | J00210 | receptor, soluble, IFN-1, IFNa-01/13 |
| 7 | 184.15 | V00540 | receptor, soluble, IFN-1, IFNa-21 |
| 8 | 175.4 | V00542 | receptor, soluble, IFN-1, IFNa-14 |
| 9 | 170.84 | V00541 | receptor, soluble, IFN-1, IFNa-05 frag |
| 10 | 168.56 | M28585 | receptor, soluble, IFN-1, IFNa-16 |
| 11 | 152.77 | X02958 | receptor, soluble, IFN-1, IFNa-06 |
| 12 | 149.33 | J00207 | receptor, soluble, IFN-1, IFNa-02 |
| 13 | 81.36 | AF030514 | receptor, soluble, chemokine, cxcl-11, I-TAC |
| 14 | 80.61 | M55067 | miscellaneous, p47-phox, neutrophil nadph oxidase factor-1 |
| 15 | 79.22 | U20982 | receptor, growth factor, IGF-1, IGFBP4, functionnal antagonist of IGF1 |
| 16 | 74.55 | M62403 | receptor, growth factor, IGF-1, IGFBP4, functionalantagonist of IGF1 |
| 17 | 73.78 | X72755 | receptor, soluble, chemokine, cxcl-09, Mig |
| 18 | 69.56 | U04636 | enzyme, COX-2, prostaglandin-endoperoxide synthase 2 |
| 19 | 67.61 | J00207 | receptor, soluble, IFN-1, IFNa-02 |
| 20 | 55.58 | U83981 | apoptosis, MYD116, GADD34 |
| 21 | 53.18 | U27467 | apoptosis, BFL-1, retards apoptosis induced by il-3deprivation |
| 22 | 46.82 | M21121 | receptor, soluble, chemokine, ccl-05 RANTES |
| 23 | 44.72 | J04130 | receptor, soluble, chemokine, ccl-04, MIP-1B |
| 24 | 41.85 | U57646 | transcription, zinc finger, CSRP2, cytoskeletal remodeling? |
| 25 | 37.04 | U12767 | transcription, nuclear receptor, orphan, MINOR |
| 26 | 34.98 | S79639 | housekeeping, EXT-1, golgi, synthesis of heparan sulfate |
| 27 | 32.89 | S79639 | housekeeping, EXT-1, golgi, synthesis of heparan sulfate |
| 28 | 32.07 | M16441 | receptor, soluble, cytokine, TNFB |
| 29 | 31.41 | D12614 | receptor, soluble, cytokine, TNFB |
| 30 | 31.16 | X02530 | receptor, soluble, chemokine, cxcl-10, IP-10, IFN responsive |
| 31 | 30.53 | U03398 | surface marker, 4-1BB ligand, CD137 interaction, costimulation |
| 32 | 29.69 | X60592 | surface marker, CD040, signaling |
| 33 | 29.21 | AI865431 | surface marker, CD040, frag? |
| 34 | 27.32 | X04430 | receptor, soluble, cytokine, IL-06, precursor, IFN, IFNb2a |
| 35 | 26.29 | AF078096 | transcription, FXC1, forkhead box protein c1 |
| 36 | 26.11 | M21121 | receptor, soluble, chemokine, ccl-05, RANTES |
| 37 | 26.04 | M16441 | receptor, soluble, cytokine, TNFB |
| 38 | 25.12 | U19261 | signaling, TRAF-1, TNF receptor-associated factor 1 |
| 39 | 24.78 | U12767 | transcription, nuclear receptor, orphan, MINOR |
| 40 | 24.42 | L31584 | receptor, surface, chemokine, ccr-07, EBI-1 |
| 41 | 23.32 | D90144 | receptor, soluble, chemokine, CCL-03, MIP-1A |
| 42 | 23.23 | AJ225089 | enzyme, OASL, 2′-5′oligoadenylate synthetase-like |
| 43 | 21.44 | U19261 | signaling, TRAF1 |
| 44 | 21.13 | D13891 | transcription, HLH, inhibitor of DNA binding 2 |
| 45 | 20.93 | D78579 | transcription, nuclear receptor, orphan, MINOR |
| 46 | 20.27 | X02910 | receptor, soluble, cytokine, TNFA |
| 47 | 17.97 | AF002986 | receptor, surface, H963, platelet activating receptor homolog |
| 48 | 17.47 | M36820 | receptor, soluble, chemokine, cxcl-02, Mip2a, GRObeta |
| 49 | 17.21 | M14660 | miscellaneous, IFN, GARG-39, IFIT2 |
| 50 | 16.78 | D14497 | kinase, MAP3K8, mitogen-activated protein kinase kinase kinase 8, cot |
| 51 | 15.83 | AF077346 | IL-18RAP, interleukin 18 receptor accessory protein |
| 52 | 15.22 | M14660 | miscellaneous, IFN, GARG-39, IFIT2 |
| 53 | 15.11 | X75042 | transcription, NF-kB, rel, v-rel |
| 54 | 14.87 | Z30644 | channel, clc-k2, chloride channel protein clc-kb |
| 55 | 14.56 | D78579 | transcription, nuclear receptor, orphan, MINOR |
| 56 | 14.55 | L11329 | phosphatase, DUS2, dual specificity protein phosphatase 2 |
| 57 | 12.47 | J05008 | receptor, ENDOTHELIN-1 PRECURSOR (ET-1), vasoconstriction |
| 58 | 12.31 | AF026939 | IFT4, Cig-49, interferon-induced protein with tetratricopeptide repeats 4 |
| 59 | 12.23 | L19871 | ATF3, CYCLIC-AMP-DEPENDENT TRANSCRIPTION FACTOR |
| 60 | 11.95 | AF005775 | apoptosis, CFLA, cellular flice-like inhibitory protein (c-flip) |
| 61 | 11.48 | AB002344 | unknown |
| 62 | 11.18 | M56803 | transcription, NF-kB, p105, nuclear factor nf-kappa-b p105 subunit |
| 63 | 11.1 | M29039 | transcription, JUN-B, transcription factor jun-b |
| 64 | 10.93 | S76638 | transcription, NF-kB, p50, (p49/p100) |
| 65 | 10.85 | M69043 | transcription, NF-kB, IkB, MAD |
| 66 | 10.56 | M15330 | receptor, soluble, cytokine, IL-01B, IL-1 beta |
| 67 | 10.55 | X61498 | transcription; NF-kB, nuclear factor nf-kappa-b p100 subunit |
| 68 | 10.47 | X58072 | transcription, GATA-3 |
| | | | ENPP2, ectonucleotide pyrophosphatase/phosphodiesterase 2 |
| 69 | 10.21 | D45421 | (autotaxin) |
| 70 | 10.17 | Z14138 | transcription, MAP3K8, mitogen-activated protein kinase kinase kinase 8 |
| 71 | 9.93 | U40992 | heat shock, DNAJB4, DnaJ (Hsp40) homolog, subfamily B, member 4 |
| 72 | 9.83 | U70426 | signaling, G protein, RGSG16, regulator of g-protein signaling 16 (rgs16) |
| 73 | 9.73 | S76638 | transcription, NF-kB, p50, (p49/p100) |
| 74 | 9.67 | AB007858 | enzyme, 5′cap guanine-N-7 methyltransferase af067791 |
| 75 | 9.65 | U45878 | apoptosis, BIR3, inhibitor, binds Traf-1 and 2 |
| 76 | 9.51 | S59049 | signalling, G protein, RGS1, regulator of g-protein signaling 1 |
| 77 | 9.46 | X07743 | signalling, pleckstrin, p47 |
| 78 | 9.21 | D13891 | HLH, inhibitor of DNA binding 2 |
| 79 | 9.09 | L40387 | OASL, 2′-5′oligoadenylate synthetase-like, nuclear receptor, TRIP14 |
| 80 | 8.89 | X89750 | TGIF, TG-interacting factor, inhibitors retinoid x receptor (rxr) |
| 81 | 8.78 | AB004904 | transcription, STAT, SOC53, STAT induced STAT inhibitor 3 |
| 82 | 8.74 | Z22576 | surface marker, CD069, C-type lectin, signaling |
| 83 | 8.64 | U77735 | kinase, pim-2, (serine threonine kinase) |
| 84 | 8.21 | AF078077 | apoptosis, GADD45B, MyD118 |
| 85 | 7.48 | M58603 | transcription, NF-kB, p105, nuclear factor nf-kappa-b p105 subunit |
| 86 | 7.43 | M36067 | replication, DNA ligase 1, ATP dependent |
| 87 | 7.33 | M16750 | signalling, kinase, pim-1 |
| 88 | 7.25 | AB002344 | unknown |
| 89 | 7.2 | L28175 | receptor, PE24 prostaglandin E receptor 4 (subtype EP4) |
| 90 | 7.11 | U49187 | miscellaneous |
| 91 | 6.97 | M24398 | transcription, parathymosin, inhibitor |
| 92 | 6.57 | AF005775 | apoptosis, CFLA |
| 93 | 6.25 | U40992 | heat shock, DNAJB4, DnaJ (Hsp40) homolog, subfamily B, member 4 |
| 94 | 6.14 | L25124 | receptor, PE24 prostaglandin E receptor 4 (subtype EP4) |
| | | | nuclear receptor, TR3 (NGFI-B, Nur77), steroid/thyroid receptor |
| 95 | 6.13 | L13740 | superfamily |
| 96 | 6.12 | Z11697 | surface marker, CD083, blast marker for DC |
| 97 | 6.11 | AF001434 | receptor, EHD1, participating in clathrin-coated pit-mediated endocytosis |
| 98 | 6.01 | AF117829 | signalling, RIPK2, receptor-interacting serine-threonine kinase 2 |
| 99 | 6 | Y11306 | TCF-4, TCF7L2 transcription factor 7-like 2 (T-cell specific, HMG-box) |
| 100 | 5.98 | S76792 | surface marker, CD134, OX40 |
| 101 | 5.98 | U91512 | surface marker, adhesion, ninjurin (nerve injury-induced protein 1) |
| 102 | 5.95 | AB000734 | signalling, SSI1, STAT-induced STAT inhibitor-1, JAK binding protein |
| 103 | 5.86 | D64142 | replication, transcription, histone, H1Fx |
| 104 | 5.74 | M92357 | TNFAIP2 tumor necrosis factor alpha-induced protein 2, RA |
| | | | induced, B94 |
| 105 | 5.72 | Z23115 | apoptosis, bcl-xL, dominant regulator of apoptotic cell death |
| | | | neuromedin B, Bombesin-like peptides, bombesin/neuromedin |
| 106 | 5.68 | AI985272 | b/ranatensin |
| 107 | 5.63 | M58603 | transcription, NF-kB, p105, nuclear factor nf-kappa-b p105 subunit |
| 108 | 5.62 | M54915 | signalling, kinase, pim-1 |
| 109 | 5.47 | Z23115 | apoptosis, bcl-xL, dominant regulator of apoptotic cell death |
| | | | transcription, nuclear, CREM, cAMP responsive element modulator, fos |
| 110 | 5.45 | S68134 | jun |
| | | | transcription, RNA pol, RPC62 polymerase (RNA) III (DNA directed) |
| 111 | 5.43 | U93867 | (62 kD) |
| 112 | 5.4 | AI971169 | unknown |
| 113 | 5.38 | AB006624 | unknown |
| 114 | 5.27 | W27419 | NT_004511.4\|Hs1_4668 Homo sapiens |
| 115 | 5.19 | M24283 | surface marker, CD054, ICAM-1 |
| 116 | 5.05 | U00672 | receptor, surface, cytokine, IL-10R |
| 117 | 4.99 | U83115 | miscellaneous, AIM1, absent in melanoma 1 |
| 118 | 4.94 | M11186 | receptor, oxytocin, prepro- (neurophysin I), contraction |
| | | | signalling, CHML, Rab escort protein-2, activating |
| 119 | 4.85 | X64728 | geranylgeranyltransferase A |
| 120 | 4.69 | W28729 | unknown |
| 121 | 4.63 | AI138605 | miscellaneous, DKFZP566A1524 hypothetical protein DKFZp566A1524 |
| 122 | 4.62 | AF030107 | signalling, G protein, RGS13, regulator of G-protein signalling 13 |
| 123 | 4.46 | X70326 | surface marker, adhesion, MacMarcks, integrin activation |
| | | | transcription, nuclear, CREM, cAMP responsive element modulator, fos |
| 124 | 4.43 | S68134 | jun |
| 125 | 4.41 | U03057 | structural protein, fascin, actin bundling protein |

TABLE 2b
Top 125 positively upmodulated genes by CpG-DNA (8 hours) in human pDC

| Rank | Sort score | Accession # | Gene Name, function |
|---|---|---|---|
| 1 | 140.66 | X02956 | receptor, soluble, IFN-1, IFNa-05 |
| 2 | 119.55 | V00540 | receptor, soluble, IFN-1, IFNa-21 |
| 3 | 118.92 | V00551 | receptor, soluble, IFN-1, IFNa-10 |
| 4 | 118.44 | M28585 | receptor, soluble, IFN-1, IFNa-16 |
| 5 | 111.7 | J00210 | receptor, soluble, IFN-1, IFNa-01/13 |
| 6 | 100.21 | M27318 | receptor, soluble, IFN-1, IFNa-04b |
| 7 | 95.47 | V00542 | receptor, soluble, IFN-1, IFNa-14 |
| 8 | 90.95 | X02958 | receptor, soluble, IFN-1, IFNa-06 |
| 9 | 89.63 | J00207 | receptor, soluble, IFN-1, IFNa-02 |
| 10 | 87.72 | M21121 | receptor, soluble, chemokine, ccl-05 RANTES |
| 11 | 86 | X04430 | receptor, soluble, cytokine, IL-06, precursor, IFN, IFNb2a |
| 12 | 84.97 | V00541 | receptor, soluble, IFN-1, IFNa-05 frag |
| 13 | 81.96 | V00535 | receptor, soluble, IFN-1, IFNb-01 |
| 14 | 74.19 | AF030514 | receptor, soluble, chemokine, cxcl-11, I-TAC |
| | | | receptor, growth factor, IGF-1, IGFBP4, functionalantagonist of |
| 15 | 66.4 | M62403 | IGF1 |
| | | | receptor, growth factor, IGF-1, IGFBP4, functionnal antagonist of |
| 16 | 61.81 | U20982 | IGF1 |
| 17 | 56.93 | X02530 | receptor, soluble, chemokine, cxcl-10, IP-10, IFN responsive |
| 18 | 51.85 | J04130 | receptor, soluble, chemokine, ccl-04, MIP-1B |
| 19 | 50.22 | X58822 | receptor, soluble, IFN-1, IFN-omega-1 |
| 20 | 48.34 | M21121 | receptor, soluble, chemokine, ccl-05, RANTES |
| 21 | 45.83 | L31584 | receptor, surface, chemokine, ccr-07, EBI-1 |
| 22 | 43.39 | M14660 | miscellaneous, IFN, GARG-39, IFIT2 |
| 23 | 41.35 | Z23115 | apoptosis, bcl-xL, dominant regulator of apoptotic cell death |
| 24 | 39.63 | AL049250 | miscellaneous, FLJ20538, BANP homolog, SMAR1 homolog |
| | | | neuromedin B, Bombesin-like peptides, bombesin/neuromedin |
| 25 | 38.98 | AI985272 | b/ranatensin |
| | | | transcription, B-ATF, basic leucine zipper transcription factor, ATF- |
| 26 | 37.32 | AF016898 | like |
| 27 | 36.79 | M14660 | miscellaneous, IFN, GARG-39, IFIT2 |
| 28 | 34.46 | U03398 | surface marker, 4-1BB ligand, CD137 interaction, costimulation |
| 29 | 33.46 | Z23115 | apoptosis, bcl-xL, dominant regulator of apoptotic cell death |
| 30 | 33.39 | AA461365 | signalling, RAB4B |
| 31 | 32.49 | D90144 | receptor, soluble, chemokine, CCL-03, MIP-1A |
| 32 | 31.42 | X75042 | transcription, NF-kB, rel, v-rel |
| 33 | 30.93 | AF098641 | surface marker, CD044RC |
| 34 | 28.55 | J00207 | receptor, soluble, IFN-1, IFNa-02 |
| 35 | 28.55 | U77735 | transcription, pim-2 |
| 36 | 27.41 | M11717 | heat shock, HSP70, heat shock 70 kD protein 1A |
| 37 | 26.81 | M59830 | heat shock, HSP70, heat shock 70 kD protein 1B, HSP-70-2 |
| 38 | 26.5 | U18671 | transcription, STAT3 |
| 39 | 25.78 | S79639 | housekeeping, EXT-1, golgi, synthesis of heparan sulfate |
| 40 | 25.17 | U27467 | apoptosis, BFL-1, retards apoptosis induced by il-3deprivation |
| 41 | 24.72 | L08096 | surface marker, CD070, receptor, TNF, CD27L |
| 42 | 24.32 | L05424 | surface marker, CD044, homing, signaling |
| 43 | 23.05 | M55067 | miscellaneous, p47-phox, neutrophil nadph oxidase factor-1 |
| 44 | 22.77 | S79639 | housekeeping, EXT-1, golgi, synthesis of heparan sulfate |
| 45 | 22.4 | L40387 | OASL, 2′-5′oligoadenylate synthetase-like, nuclear receptor, TRIP14 |
| 46 | 22.22 | X72755 | receptor, soluble, chemokine, cxcl-09, Mig |
| 47 | 21.9 | W25936 | GCN5L2, transcriptional activator, histone acetyltransferase activity |
| 48 | 21.6 | D84276 | surface marker, CD038, ADP-ribosyl cyclase 1, signaling |
| 49 | 21.55 | AE005775 | apoptosis, CFLA |
| 50 | 21.44 | AD000092 | enzyme, GCDH, glutaryl-coa dehydrogenase, mitochondrial |
| 51 | 21.14 | D37965 | receptor, PDGFRL, platelet-derived growth factor receptor-like |
| | | | IFT4, Cig-49, interferon-induced protein with tetratricopeptide |
| 52 | 20.91 | AF026939 | repeats 4 |
| 53 | 20.87 | K01383 | enzyme, anti-oxidative stress, MT1A, metallothionein-ia (mt-1a). |
| 54 | 20.2 | Z12173 | proteolysis, lysosomal, GL6S, n-acetylglucosamine-6-sulfatase |
| 55 | 20.04 | X67325 | CDK inhibitor, p27, interferon-alpha induced 11.5 kda protein (p27) |
| 56 | 20 | X60592 | surface marker, CD040, signaling |
| 57 | 19.88 | M59040 | surface marker, CD044, homing, signaling |
| 58 | 19.56 | AJ225089 | enzyme, OASL, 2′-5′oligoadenylate synthetase-like |
| 59 | 19.29 | M16441 | receptor, soluble, cytokine, TNFB |
| 60 | 18.82 | U71364 | miscellaneous, SERPINB9, serine (or cysteine) proteinase inhibitor, |
| 61 | 18.2 | AF055022 | unknown |
| 62 | 18.14 | U16031 | transcription, STAT6, activated by IL-4 and IL-13 |
| 63 | 18.03 | U57646 | transcription, zinc finger, CSRP2, cytoskeletal remodeling? |
| 64 | 17.8 | HG4322- | structural protein, tubulin |
| | | HT4592 | |
| 65 | 17.35 | U18671 | transcription, STAT2 |
| 66 | 17.19 | AL050028 | unknown |
| 67 | 16.46 | W25921 | proteolysis, lysosomal, GL6S, n-acetylglucosamine-6-sulfatase |
| 68 | 16.43 | X79535 | structural protein, tubulin, beta polypeptide, TUBB |
| 69 | 16.33 | Z12173 | proteolysis, lysosomal, GL6S, n-acetylglucosamine-6-sulfatase |
| 70 | 16.3 | U04636 | enzyme, COX-2, prostaglandin-endoperoxide synthase 2 |
| 71 | 15.98 | M28130 | receptor, soluble, chemokine, cxcl-08, interleukin-8 precursor |
| 72 | 15.58 | AC004528 | miscellaneous, hypothetical protein R32184_1 |
| 73 | 15.57 | L09235 | ATPase, lysosomal, H+ transporting (vacuolar proton pump), |
| 74 | 15.34 | HG4322- | structural protein, tubulin |
| | | HT4592 | phosphatase, PTP1B, protein tyrosine phosphatase, non-receptor |
| 75 | 15.14 | M33684 | type 1 |
| 76 | 14.88 | AF015451 | apoptosis, CFLA |
| 77 | 14.59 | AL050374 | unknown |
| 78 | 14.56 | U20816 | NFKB2 nuclear factor of kappa light polypeptide gene (p49/p100) |
| 79 | 14.54 | U72206 | G protein, rho/rac guanine nucleotide exchange factor (GEF) 2 |
| 80 | 14.41 | AF055000 | unknown |
| 81 | 14.4 | U12767 | transcription, nuclear receptor, orphan, MINOR |
| 82 | 14.37 | U19261 | signaling, TRAF1 |
| 83 | 14.16 | L05424 | surface marker, CD044, homing, signaling |
| 84 | 14.05 | L25124 | receptor, PE24 prostaglandin E receptor 4 (subtype EP4) |
| 85 | 14.03 | X15334 | kinase, CKB, creatine kinase, b chain |
| 86 | 13.47 | D78579 | transcription, nuclear receptor, orphan, MINOR |
| 87 | 13.4 | AF005775 | apoptosis, CFLA, cellular flice-like inhibitory protein (c-flip) |
| 88 | 13.39 | X75042 | transcription, NF-kB, c-rel proto-oncogene protein (c-rel protein) |
| 89 | 13.26 | M83667 | NF-IL6b, ccaat/enhancer binding protein delta (c/ebp delta), C/EBP |
| 90 | 13.24 | U77735 | kinase, pim-2, (serine threonine kinase) |
| 91 | 13.15 | M36820 | receptor, soluble, chemokine, cxcl-02, Mip2a, GRObeta |
| 92 | 12.94 | L08599 | E-cadherin, epithelial-cadherin |
| | | | elastase inhibitor, SERPINB1, serine (or cysteine) proteinase |
| 93 | 12.56 | M93056 | inhibitor |
| 94 | 12.47 | AI671547 | signalling, RAB9, member RAS oncogene family |
| 95 | 12.45 | M19650 | signalling, CNP, 2′,3′-cyclic nucleotide 3′-phosphodiesterase |
| 96 | 12.4 | L78440 | transcription, STAT4, IL-12 signalling |
| 97 | 12.23 | M29335 | MHC-DN-alpha, co-chaperone of HLA-DM in peptide loading |
| 98 | 12.07 | L41680 | enzyme, PST, alpha-2,8-polysialyltransferase |
| 99 | 11.94 | X15334 | kinase, CKB, creatine kinase, b chain |
| 100 | 11.9 | D13891 | transcription, HLH, inhibitor of DNA binding 2 |
| 101 | 11.61 | M58603 | transcription, NF-kB, p105, nuclear factor nf-kappa-b p105 subunit |
| 102 | 11.59 | AF010312 | miscellaneous, PIG7, LPS-induced TNF-alpha factor |
| 103 | 11.51 | AF023203 | transcription, homeobox |
| 104 | 11.46 | X68742 | CD049a, integrin, alpha-1 (laminin and collagen receptor) (vla-1) |
| 105 | 11.44 | U19261 | signaling, TRAF-1, TNF receptor-associated factor 1 |
| 106 | 11.17 | M10943 | anti-oxidative stress, MT1F, metallothionein-if (mt-1f). --gene: mt1 |
| 107 | 11.06 | M80563 | placental calcium-binding protein (calvasculin) |
| 108 | 11.02 | AB000887 | receptor, soluble, chemokine, ccl-19, ELC |
| 109 | 10.76 | U43185 | transcription, STAT5A |
| 110 | 10.75 | M87284 | enzyme, 2-5A2, 2′-5′oligoadenylate synthetase 2, IFN response |
| 111 | 10.73 | AJ000480 | kinase, C8FW, phosphoprotein regulated by mitogenic pathways |
| 112 | 10.55 | X01057 | IL-02RA |
| 113 | 10.41 | L33799 | enzyme, PCO1, procollagen C-endopeptidase enhancer |
| 114 | 10.3 | AL036554 | DEFA1, defensin, alpha 1 |
| 115 | 10.26 | X68742 | CD049a, integrin, alpha-1 (laminin and collagen receptor) (vla-1) |
| 116 | 10.16 | AF026941 | unknown |
| 117 | 10.13 | U49395 | purinoceptor, P2X5a, p2x purinoceptor 5 (atp receptor) |
| 118 | 10.03 | X98248 | sortilin1, sortilin precursor (glycoprotein 95)(neurotensin receptor 3) |
| 119 | 9.92 | AF070530 | unknown, hypothetical protein, clone 24751 |
| 120 | 9.88 | U33838 | transcription, NF-kB |
| 121 | 9.76 | U90908 | miscellaneous, hypothetical protein from clones 23549 and 23762 |
| 122 | 9.69 | M27533 | CD080 |
| 123 | 9.55 | X51730 | transcription, nuclear receptor, PRGR, progesterone receptor |
| 124 | 9.29 | Y16645 | receptor, soluble, chemokine, ccl-08, MCP-2 |
| 125 | 9.1 | AB020653 | unknown |

TABLE 2c
Top 125 positively upmodulated genes by CpG-DNA (24 hours) in human pDC

| Rank | Sort score | Accession # | Gene Name, function |
|---|---|---|---|
| 1 | 65.86 | AB000887 | receptor, soluble, chemokine, ccl-19, ELC |
| 2 | 48.9 | AI381790 | unknown, adipose specific 2 (APM2), collagen-like factor |
| 3 | 47.07 | Z82244 | CDC46, dna replication licensing factor mcm5 (p1-cdc46) |
| 4 | 46.9 | L37747 | structural protein, cell cycle, lamin b1 |
| 5 | 43.71 | L31584 | receptor, surface, chemokine, ccr-07, EBI-1 |
| 6 | 43.51 | U19261 | signaling, TRAF-1, TNF receptor-associated factor 1 |
| 7 | 41.88 | U90908 | miscellaneous, hypothetical protein from clones 23549 and 23762 |
| 8 | 41.49 | X02530 | receptor, soluble, chemokine, cxcl-10, IP-10, IFN responsive |
| 9 | 41.37 | HG4322- | structural protein, tubulin |
| | | HT4592 | |
| 10 | 40.97 | X15334 | kinase, CKB, creatine kinase, b chain |
| | | | receptor, growth factor, IGF-1, IGFBP4, functionnal antagonist of |
| 11 | 39.15 | U20982 | IGF1 |
| | | | receptor, growth factor, IGF-1, IGFBP4, functionalantagonist of |
| 12 | 38.25 | M62403 | IGF1 |
| 13 | 35.71 | X79535 | structural protein, tubulin, beta polypeptide, TUBB |
| 14 | 34.06 | X15334 | kinase, CKB, creatine kinase, b chain |
| 15 | 30.44 | U19261 | signaling, TRAF1 |
| 16 | 24.08 | L08096 | surface marker, CD070, receptor, TNF, CD27L |
| 17 | 23.18 | U27467 | apoptosis, BFL-1, retards apoptosis induced by il-3deprivation |
| 18 | 23.06 | X72755 | receptor, soluble, chemokine, cxcl-09, Mig |
| 19 | 22.21 | HG4322- | structural protein, tubulin |
| | | HT4592 | |
| 20 | 20.55 | W27419 | NT_004511.4\|Hs1_4668 Homo sapiens |
| 21 | 20.41 | D78579 | transcription, nuclear receptor, orphan, MINOR |
| | | | transcription, B-ATF, basic leucine zipper transcription factor, ATF- |
| 22 | 20.36 | AF016898 | like |
| 23 | 19.97 | X15949 | transcription, IRF-2 |
| 24 | 19.65 | X04430 | receptor, soluble, cytokine, IL-06, precursor, IFN, IFNb2a |
| 25 | 19.54 | AF078077 | apoptosis, GADD45B, MyD118 |
| 26 | 19.5 | X68277 | MKP-1, DUS1, dual specificity protein phosphatase 1 |
| 27 | 18.34 | U83171 | receptor, soluble, chemokine, ccl-22, MDC |
| 28 | 18.05 | AA461365 | signalling, RAB4B |
| 29 | 17.82 | AF032906 | proteolysis, cathepsin Z, cysteine proteinase |
| 30 | 17.4 | AL050374 | unknown |
| 31 | 16.27 | U12767 | transcription, nuclear receptor, orphan, MINOR |
| 32 | 16.19 | AI671547 | signalling, RAB9, member RAS oncogene family |
| 33 | 15.33 | AJ000480 | kinase, C8FW, phosphoprotein regulated by mitogenic pathways |
| 34 | 15.13 | M58603 | transcription, NF-kB, p105, nuclear factor nf-kappa-b p105 subunit |
| 35 | 15.05 | X59871 | TCF-1, Lef, t-cell-specific transcription factor 1 (tcf-1) |
| 36 | 14.02 | Z82200 | receptor, P2Y10, putative purinergic receptor |
| 37 | 13.8 | U03398 | surface marker, 4-1BB ligand, CD137 interaction, costimulation |
| 38 | 13.67 | L25124 | receptor, PE24 prostaglandin E receptor 4 (subtype EP4) |
| 39 | 13.34 | M27533 | CD080 |
| 40 | 13.22 | D78579 | transcription, nuclear receptor, orphan, MINOR |
| 41 | 12.94 | AF030698 | CD108, H-SEMA |
| | | | elastase inhibitor, SERPINB1, serine (or cysteine) proteinase |
| 42 | 12.92 | M93056 | inhibitor |
| 43 | 12.66 | D13891 | transcription, HLH, inhibitor of DNA binding 2 |
| 44 | 11.91 | U20816 | NFKB2 nuclear factor of kappa light polypeptide gene (p49/p100) |
| 45 | 11.66 | AL050028 | unknown |
| 46 | 11.57 | AL022310 | surface marker, OX40 ligand |
| 47 | 11.49 | U15932 | phosphatase, DUS5, dual specificity protein phosphatase 5, HVH3 |
| | | | transcription, zinc finger protein 147 (estrogen-responsive finger |
| 48 | 11.44 | D21205 | protein) |
| 49 | 11.21 | Z23115 | apoptosis, bcl-xL, dominant regulator of apoptotic cell death |
| 50 | 11.2 | M69043 | transcription, NF-kB, IkB, MAD |
| 51 | 11.02 | AF030514 | receptor, soluble, chemokine, cxcl-11, I-TAC |
| 52 | 10.66 | L28175 | receptor, PE24 prostaglandin E receptor 4 (subtype EP4) |
| 53 | 10.54 | AF000545 | receptor, P2Y10, putative purinergic receptor |
| | | | receptor, TRIP-10 CIP-4, thyroid receptor interacting protein 10, |
| 54 | 10.07 | AJ000414 | ligand |
| 55 | 9.95 | AB007447 | signaling, FLN29, TRAF-interacting zinc finger protein FLN29 |
| 56 | 9.73 | X67325 | CDK inhibitor, p27, interferon-alpha induced 11.5 kda protein (p27) |
| 57 | 9.68 | X93086 | enzyme, oxidoreductase, biliverdin reductase a precursor |
| 58 | 9.66 | M29039 | transcription, JUN-B, transcription factor jun-b |
| 59 | 9.53 | AB028954 | unknown |
| 60 | 9.43 | X79067 | transcription, nuclear, ERF-1 |
| 61 | 9.35 | H68340 | transcription, RNAHP, RNA helicase-related protein |
| 62 | 9.28 | AB019517 | signalling, IPKG, protein kinase inhibitor gamma |
| 63 | 9.19 | U48263 | nociceptin precursor (orphanin fq) (ppnoc) |
| 64 | 9.12 | U16954 | protein af1q |
| 65 | 8.88 | Z11697 | surface marker, CD083, blast marker for DC |
| | | | TNFAIP2 tumor necrosis factor alpha-induced protein 2, RA induced, |
| 66 | 8.88 | M92357 | B94 |
| 67 | 8.86 | M21121 | receptor, soluble, chemokine, ccl-05 RANTES |
| 68 | 8.77 | AA418437 | miscellaneous, hypothetical protein FLJ20505 |
| 69 | 8.66 | M11717 | heat shock, HSP70, heat shock 70 kD protein 1A |
| 70 | 8.65 | X70326 | surface marker, adhesion, MacMarcks, integrin activation |
| 71 | 8.62 | M59830 | heat shock, HSP70, heat shock 70 kD protein 1B, HSP-70-2 |
| 72 | 8.5 | AJ225089 | enzyme, OASL, 2′-5′oligoadenylate synthetase-like |
| 73 | 8.05 | M58603 | transcription, NF-kB, p105, nuclear factor nf-kappa-b p105 subunit |
| 74 | 8.01 | AB028952 | structural protein, synaptopodin |
| 75 | 7.98 | Y00097 | signalling, annexin 6, one binding site for calcium and phospholipid |
| 76 | 7.94 | D37965 | receptor, PDGFRL, platelet-derived growth factor receptor-like |
| 77 | 7.94 | AI936759 | structural protein, ???, clathrin-associated/assembly/adaptor protein |
| 78 | 7.92 | M14660 | miscellaneous, IFN, GARG-39, IFIT2 |
| 79 | 7.91 | U57646 | transcription, zinc finger, CSRP2, cytoskeletal remodeling? |
| 80 | 7.86 | U43185 | transcription, STAT5A |
| 81 | 7.51 | U78575 | PIP5K1A, phosphatidylinositol-4-phosphate 5-kinase, type I, alpha |
| 82 | 7.4 | X68742 | CD049a, integrin, alpha-1 (laminin and collagen receptor) (vla-1) |
| 83 | 7.4 | X03473 | replication, transcription, histone, H10, H1 histone family, member 0 |
| 84 | 7.29 | M58603 | transcription, NF-kB, p105, nuclear factor nf-kappa-b p105 subunit |
| 85 | 7.22 | D42043 | unknown |
| 86 | 7.18 | Y10256 | NIK, MAP3K14, mitogen-activated protein kinase kinase kinase 14 |
| 87 | 7.13 | S76638 | transcription, NF-kB, p50, (p49/p100) |
| 88 | 7.07 | X70683 | transcription, SOX-4, transcription factor sox-4 |
| | | | miscellaneous, DKFZP566A1524 hypothetical protein |
| 89 | 7.05 | AI138605 | DKFZp566A1524 |
| 90 | 7.05 | AF039656 | brain acid soluble protein 1 (basp1 protein) |
| 91 | 7.01 | AB008913 | transcription, PAX-4, homeobox, cell differentiation and development |
| 92 | 6.94 | AF030196 | miscellaneous, apoptosis, stannin |
| 93 | 6.94 | X63131 | transcription, probable transcription factor pml. --gene: pml or myl. |
| 94 | 6.93 | S72869 | unknown |
| | | | phosphatase, PTP1B, protein tyrosine phosphatase, non-receptor |
| 95 | 6.85 | M33684 | type 1 |
| 96 | 6.83 | U04343 | surface marker, CD086 |
| 97 | 6.8 | AF087036 | transcription, HLH, musculin (activated B-cell factor-1) |
| 98 | 6.79 | S76638 | transcription, NF-kB, p50, (p49/p100) |
| 99 | 6.75 | U67171 | enzyme, selenoprotein w. sepw1 or selw, redo-rleated processes |
| 100 | 6.71 | X92814 | miscellaneous, similar to rat HREV107 |
| 101 | 6.68 | AB013924 | TSC403, similar to lysosome-associated membrane glycoprotein |
| 102 | 6.65 | AB022718 | miscellaneous, DEPP, decidual protein induced by progesterone |
| 103 | 6.48 | D84276 | surface marker, CD038, ADP-ribosyl cyclase 1, signaling |
| 104 | 6.45 | S81914 | IEX-1L, (TNF) radiation-inducible immediate-early |
| 105 | 6.34 | AL022165 | enzyme, carbohydrate (N-acetylglucosamine 6-O) sulfotransferase 7 |
| 106 | 6.24 | AF005775 | apoptosis, CFLA |
| 107 | 6.13 | U31628 | receptor, surface, cytokine, il-15RA |
| 108 | 6.13 | X61498 | transcription; NF-kB, nuclear factor nf-kappa-b p100 subunit |
| 109 | 6.11 | AA442560 | miscellaneous, phorbolin-like protein MDS019 |
| 110 | 6.11 | AB014553 | unknown |
| 111 | 6.09 | U12767 | transcription, nuclear receptor, orphan, MINOR |
| 112 | 5.99 | AF005775 | apoptosis, CFLA, cellular flice-like inhibitory protein (c-flip) |
| 113 | 5.89 | W25986 | miscellaneous, hypothetical protein DKFZp564K0822 |
| | | | IFT4, Cig-49, interferon-induced protein with tetratricopeptide |
| 114 | 5.87 | AF026939 | repeats 4 |
| 115 | 5.85 | Z23115 | apoptosis, bcl-xL, dominant regulator of apoptotic cell death |
| 116 | 5.78 | U71364 | miscellaneous, SERPINB9, serine (or cysteine) proteinase inhibitor, |
| 117 | 5.73 | M18533 | structural protein, dystrophin |
| 118 | 5.58 | AF027826 | |
| 119 | 5.53 | X03473 | replication, transcription, histone |
| 120 | 5.48 | AB014587 | unknown |
| 121 | 5.39 | X68149 | receptor, surface, chemokine, cxcr-05, BCA-1 |
| 122 | 5.33 | L40387 | OASL, 2′-5′oligoadenylate synthetase-like, nuclear receptor, TRIP14 |
| 123 | 5.31 | Y08319 | structural protein, kinesin-like protein kif2 (kinesin-2) (hk2) |
| 124 | 5.29 | X66435 | enzyme, 3-hydroxy-3-methylglutaryl-Coenzyme A synthase 1 |
| | | | (soluble) |
| 125 | 5.27 | AF010193 | transcription, SMAD7 |

It has been discovered according to the invention that exposure to CpG immunostimulatory nucleic acids impacts on the expression of a variety of genes. The genes discussed herein are divided into those that are induced, suppressed, upregulated, or downregulated as a result of exposure to CpG. A gene is considered induced if its expression product (e.g., mRNA or cDNA) was absent (A) in the CpG-DNA non-stimulated pDC population and became present (P) following CpG exposure. A gene is considered suppressed if it was present (P) in the CpG-DNA non-stimulated pDC population and became absent (A) following CpG exposure. A gene is considered upregulated if it was present (P) in the CpG-DNA non-stimulated pDC population, and its level of expression increased following CpG exposure. A gene is considered downregulated if it was present (P) in the CpG-DNA non-stimulated pDC population, and it was present but at a reduced level of expression following CpG exposure.

TABLE 3a
Cell Surface Markers Induced at 2 Hours of CpG Exposure

| Rank | Accession # | Gene name |
|---|---|---|
| 1 | AF004231 | ILT4 |
| 2 | L08096 | CD070 |
| 3 | S71043 | CD079a |
| 4 | X59770 | CD121b |

No transcripts were determined to be suppressed at 2 hours of CpG exposure.

TABLE 3b
Cell Surface Markers Upregulated at 2 Hours of CpG Exposure

| Rank | Accession # | Gene name |
|---|---|---|
| 1* | U03398 | 4-1BB ligand, CD137 interaction, |
| | | costimulation |
| 2* | X60592 | CD040, signaling |
| 3* | Z22576 | CD069, C-type lectin, signaling |
| 4 | Z11697 | CD083, blast marker for DC |
| 5 | U91512 | adhesion, ninjurin (nerve injury-induced |
| | | protein 1) |
| 6 | S76792 | CD134, OX40 |
| 7 | M24283 | CD054, ICAM-1 |
| 8 | X70326 | adhesion, MacMarcks, integrin activation |
| 9 | M37766 | CD048, ligand CD2 |
| 10 | HG371-HT26388 | mucin |
| 11 | D11086 | CD-132, IL-2RG |
| 12 | J05581 | CD227, MUC-1, mucin 1 |
| 13 | AF098641 | CD044RC |
| 14 | X14046 | CD037, 4TM B cell signaling |

Those markers shown in shaded cells (and/or with asterisks “*”) throughout these tables were observed to have at least 10-fold changes relative to the resting state.

TABLE 3c
Cell Surface Markers Downregulated at 2 Hours of CpG Exposure

| Rank | Accession # | Gene name |
|---|---|---|
| 1 | D83597 | CD180, RP-105, TLR4, LPS, B cells |
| 2 | J02973 | CD141, thrombomodulin |
| 3 | U19247 | CD119, IFNG-Ra |
| 4 | AF012023 | integrin cytoplasmic domain associated protein |
| | | (Icap-1a) |
| 5 | Z50022 | pituitary tumor-transforming 1 interacting protein |

TABLE 4a
Cell Surface Markers Induced at 8 Hours of CpG Exposure

| Rank | Accesion # | Gene name |
|---|---|---|
| 1* | L08096 | CD070, receptor, TNF, CD27L |
| 2* | L05424 | CD044, homing, signaling |
| 3* | X68742 | CD049a, integrin, alpha-1, (vla-1) |
| 4 | AF004231 | ILT4 |
| 5 | M35011 | integrin beta-5 subunit |
| 6 | S71043 | CD079a |
| 7 | M35093 | CD227, MUC-1, mucin 1 |
| 8 | M12807 | CD004 |

TABLE 4b
Cell Surface Markers Suppressed at 8 Hours of CpG Exposure

| Rank | Accession # | Gene name |
|---|---|---|
| 1* | M60922 | flotillin 2, involved in cell adhesion |
| 2 | L34657 | CD031, PECAM-1, diapedesis |
| 3 | J03779 | CD010 |
| 4 | M28827 | CD001c |
| 5 | AF030698 | CD108, H-SEMA, ligand for plexin C1 |
| 6 | M28170 | CD019 |

TABLE 4c
Cell Surface Markers Upregulated at 8 Hours of CpG Exposure

| Rank | Accession # | Gene name |
|---|---|---|
| 1* | U03398 | 4-1BB ligand, CD137 interaction, |
| | | costimulation |
| 2* | AF098641 | CD044RC |
| 3* | L05424 | CD044, homing, signaling |
| 4* | D84276 | CD038, ADP-ribosyl cyclase 1, signaling |
| 5* | X60592 | CD040 signaling |
| 6* | X68742 | CD049a, integrin, alpha-1 (vla-1) |
| 7 | M27533 | CD080 |
| 8 | U91512 | ninjurin (nerve injury-induced protein 1) |
| 9 | L05424 | CD044, homing, signaling |
| 10 | Z11697 | CD083, blast marker for DC |
| 11 | U66711 | LY6E |
| 12 | M24283 | CD054, ICAM-1 |
| 13 | U04343 | CD086 |
| 14 | HG3477-HT3670 | CD004 |
| 15 | AB006782 | galectin 9, galactoside-binding, chemoattractant |
| 16 | D28137 | BST-2, (bone marrow stromal antigen 2) |
| 17 | AF044968 | CD112, poliovirus receptor related 2 |
| 18 | U52112 | CD171 |
| 19 | AF025527 | ILT6, LIR-4 |
| 20 | U87947 | (emp-3) (ymp protein) |
| 21 | X13403 | CD014 |
| 22 | M58597 | CD015, ELAM-1 |
| 23 | X70326 | MacMarcks, integrin activation |
| 24 | Z22576 | CD069, C-type lectin, signaling |
| 25 | U03397 | CD137, 4-1BB |

TABLE 4d
Cell Surface Markers Downregulated at 8 Hours of CpG Exposure

| Rank | Accession # | Gene name |
|---|---|---|
| 1* | U25956 | CD162, p-selectin glycoprotein ligand 1 precursor |
| | | (psgl-1) |
| 2* | M80244 | CD098 associated, glycoprotein 4F2 light chain |
| 3* | M12886 | TCRB, T-cell receptor, beta cluster |
| 4* | U09937 | CD087, urokinase plasminogen activator surface |
| | | receptor |
| 5* | M60922 | flotillin 2, involved in cell adhesion |
| 6* | N90866 | CD052, antibody extremely lytic |
| 7* | M32315 | CD120b, TNF-R2, p80 (p75) |
| 8 | L34657 | CD031, PECAM-1, diapedesis |
| 9 | X67301 | Ig-M constant, Immunoglobin heavy constant mu |
| 10 | L06797 | CD184, SDF R, chemokine, CXCR-04, G protein |
| 11 | L34657 | CD031, PECAM-1, diapedesis |
| 12 | M93221 | CD206, lectin, C-type, mrc1, macrophage mannose |
| | | receptor |
| 13 | J02973 | CD141, thrombomodulin |
| 14 | J03779 | CD010 |
| 15 | M38690 | CD009 |
| 16 | M15395 | CD018, beta-2 integrin |
| 17 | X57809 | Ig-L, Immunoglobulin lambda locus |
| 18 | M23197 | CD033, sialoadhesin |
| 19 | M29696 | CD127, IL-7RA |
| 20 | AJ223183 | receptor, DORA protein |
| 21 | AF041261 | ILT7 |
| 22 | M28827 | CD001c |
| 23 | Y00062 | CD045 |
| 24 | AF030698 | CD108, H-SEMA, ligand for plexin C1 |
| 25 | AF004230 | ILT2 |
| 26 | M68892 | integrin B7 |
| 27 | X16983 | CD049d, integrin, alpha4 |
| 28 | X72012 | CD105 |
| 29 | X62744 | MHC-II, HLA-DMA |
| 30 | M28170 | CD019 |

TABLE 5a
Cell Surface Markers Induced at 24 Hours of CpG Exposure

| Rank | Accession # | Gene name |
|---|---|---|
| 1* | L08096 | CD070, receptor, CD27L |
| 2* | AF030698 | CD108, H-SEMA, ligand for plexin C1 |
| 3* | AL022310 | OX40 ligand |
| 4 | X68742 | CD049a, integrin, alpha-1 (vla-1) |
| 5 | X59350 | CD022, SIGLEC, sialoadhesin, B cell signaling |
| 6 | HG2147-HT2217 | mucin-3 |
| 7 | M58597 | CD015, ELAM-1 |

TABLE 5b
Cell Surface Markers Suppressed at 24 Hours of CpG Exposure

| Rank | Accession # | Gene name |
|---|---|---|
| 1* | M93221 | CD206, macrophage mannose receptor |
| 2 | AJ223183 | receptor, DORA protein |
| 3 | M23197 | CD033, sialoadhesin |

TABLE 5c
Cell Surface Markers Upregulated at 24 Hours of CpG Exposure

| Rank | Accession # | Gene name |
|---|---|---|
| 1* | U03398 | 4-1BB ligand, CD137 interaction, costimulation |
| 2* | M27533 | CD080 |
| 3 | Z11697 | CD083, blast marker for DC |
| 4 | X70326 | MacMarcks, integrin activation |
| 5 | U04343 | CD086 |
| 6 | D84276 | CD038, ADP-ribosyl cyclase 1, signaling |
| 7 | U02687 | CD135, FLT3, STK-1 |
| 8 | U91512 | ninjurin (nerve injury-induced protein 1) |
| 9 | X60592 | CD040, signaling |
| 10 | Y00636 | CD058, LFA-3, ligand for CD2 |
| 11 | D26579 | CD156, metaloprotease, ADAM8, extravasation |
| 12 | AF011333 | CD205, DEC-205 |
| 13 | AF044968 | CD112, poliovirus receptor related 2 |
| 14 | M68892 | integrin B7 |
| 15 | AF098641 | CD044RC |
| 16 | U66711 | LY6E |
| 17 | U48705 | CD167a, DDR1, tyrosine kinase receptors |
| 18 | Z49107 | galectin 9, galactoside-binding, chemoattractant |

TABLE 5d
Cell Surface Markers Downregulated at 24 Hours of CpG Exposure

| Rank | Accession # | Gene name |
|---|---|---|
| 1* | AF041261 | ILT7 |
| 2* | N90866 | CD052 |
| 3* | AF004230 | ILT2 |
| 4* | M93221 | CD206, mrc1, macrophage mannose receptor |
| 5* | X74039 | CD087 |
| 6* | M59941 | CD-131 |
| 7* | X95876 | CD183, IP10/Mig R, CXCR-03 |
| 8* | HG3477-HT3670 | CD004 |
| 9* | X14046 | CD037, 4TM B cell signaling |
| 10 | M15395 | CD018, beta-2 integrin |
| 11 | M80244 | CD098 associated, 4F2 light chain |
| 12 | X67301 | Ig-M constant, constant mu |
| 13 | AJ223183 | receptor, DORA protein |
| 14 | M16279 | CD099, mucin |
| 15 | M16336 | CD002 |
| 16 | M12886 | TCRB, T-cell receptor, beta cluster |
| 17 | AF072099 | ILT3 |
| 18 | X16983 | CD049d, integrin, alpha4 |
| 19 | U52112 | CD171 |
| 20 | AF013249 | ILT-1 |
| 21 | M37033 | CD053 |
| 22 | M25280 | CD062L, L-selectin, (lymph node homing |
| | | receptor) |
| 23 | L34657 | CD031, PECAM-1, diapedesis |
| 24 | AJ010099 | NKp44, activating NK-receptor |
| 25 | J02939 | CD098, regulation of activation |
| 26 | S76792 | CD134, OX40 |
| 27 | D14043 | CD164 MUC-24, sialomucin |
| 28 | M37766 | CD048, ligand CD2 |
| 29 | M23197 | CD033, sialoadhesin |
| 30 | Y14768 | NKp30, NK cell receptor, activation, killing |
| 31 | X74328 | integrin, alpha7b |
| 32 | J02973 | CD141, thrombomodulin |
| 33 | X62822 | CD075 |
| 34 | AF012023 | integrin, Icap-1a |
| 35 | Z50022 | pituitary tumor-transforming 1 interacting |
| | | protein |
| 36 | M32315 | CD120b, TNF-R2, p80 (p75) |

TABLE 6a
Cytokines and Chemokines and Receptors
Induced at 2 Hours of CpG Exposure

| Rank | Accession # | Gene name |
|---|---|---|
| 1* | X02956 | IFN-1, IFNa-05 |
| 2* | V00535 | IFN-1, IFNb-01 |
| 3* | X02958 | IFN-1, IFNa-06 |
| 4* | X72755 | chemokine, cxcl-09, Mig |
| 5* | J00207 | IFN-1, IFNa-02 |
| 6* | M21121 | chemokine, ccl-05, RANTES |
| 7* | AF077346 | cytokine, IL-18RAP |
| 8 | AB000887 | chemokine, ccl-19, ELC |
| 9 | Y13710 | chemokine, ccl-18, DC-CK-1, PARC, MIP-4 |

No significant suppression was observed in cytokine or chemokines or their receptors at 2 hours.

TABLE 6b
Cytokines and Chemokines and Receptors
Upregulated at 2 Hours of CpG Exposure

| Rank | Accession # | Gene name |
|---|---|---|
| 1* | L31584 | chemokine, ccr-07, EBI-1 |
| 2 | U00672 | cytokine, IL-10R |
| 3 | L08177 | chemokine, EBI2, ebv-induced g protein-coupled |
| | | receptor 2 |
| 4 | U43672 | cytokine, IL-18R1, Interleukin 18 receptor 1, |
| 5 | U31628 | cytokine, il-15RA |
| 6 | AF072902 | cytokine, IL-06R, gp130, signalling |

TABLE 6c
Cytokines and Chemokines and Receptors
Downregulated at 2 Hours of CpG Exposure

| Rank | Accession # | Gene name |
|---|---|---|
| 1 | D50683 | cytokine, TGFBR2, transforming growth factor beta |
| | | receptor II |

TABLE 7a
Cytokines and Chemokines and Receptors
Induced at 8 Hours of CpG Exposure

| Rank | Accession # | Gene name |
|---|---|---|
| 1* | X02958 | IFN-1, IFNa-06 |
| 2* | V00541 | IFN-1, IFNa-05 |
| 3* | M21121 | chemokine, ccl-05, RANTES |
| 4* | X72755 | chemokine, cxcl-09, Mig |
| 5* | X01057 | cytokine, IL-02RA, interleukin-2 receptor alpha |
| 6 | AF035279 | cytokine, il-15RA |
| 7 | M65291 | cytokine, IL-12a, p35 |
| 8 | AF077346 | cytokine, IL-18RAP |
| 9 | U86358 | chemokine, ccl-25, TECK |

TABLE 7b
Cytokines and Chemokines and Receptors
Suppressed at 8 Hours of CpG Exposure

| Rank | Accession # | Gene name |
|---|---|---|
| 1* | U95626 | chemokine, ccr-05, mip-1-alpha, mip-1-beta and |
| | | rantes |
| 2 | U03905 | chemokine, ccr-02 |

TABLE 7c
Cytokines and Chemokines and Receptors
Upregulated at 8 Hours of CpG Exposure

| Rank | Accession # | Gene name |
|---|---|---|
| 1* | X02956 | IFN-1, IFNa-05 |
| 2* | V00551 | IFN-1, IFNa-10 |
| 3* | V00535 | IFN-1, IFNb-01 |
| 4* | L31584 | chemokine, ccr-07, EBI-1 |
| 5* | J00207 | IFN-1, IFNa-02 |
| 6* | AB000887 | chemokine, ccl-19, ELC |
| 7 | Y16645 | chemokine, ccl-08, MCP-2 |
| 8 | U31628 | cytokine, il-15RA |
| 9 | AF072902 | cytokine, IL-06R, gp130, signalling |

TABLE 7d
Cytokines and Chemokines and Receptors
Downregulated at 8 Hours of CpG Exposure

| Rank | Accession # | Gene name |
|---|---|---|
| 1* | D50683 | cytokine, TGFBR2, tgf-beta receptor type ii |
| | | precursor |
| 2* | U20350 | chemokine, CX3CR-01 |
| 3 | U00672 | cytokine, IL-10R |
| 4 | D43767 | chemokine, ccl-17, TARC |
| 5 | U95626 | chemokine, ccr-05, mip-1-alpha, mip-1-beta and |
| | | rantes |

TABLE 8a
Cytokines and Chemokines and Receptors
Induced at 24 Hours of CpG Exposure

| Rank | Accession # | Gene name |
|---|---|---|
| 1* | X72755 | chemokine, cxcl-09, Mig |
| 2 | X68149 | chemokine, cxcr-05, BCA-1 |
| 3 | AF035279 | cytokine, il-15RA |
| 4 | M21121 | chemokine, ccl-05, RANTES |
| 5 | X01057 | cytokine, IL-02RA, interleukin-2 receptor alpha |
| | | chain |

TABLE 8b
Cytokines and Chemokines and Receptors
Suppressed at 24 Hours of CpG Exposure

| Rank | Accession # | Gene name |
|---|---|---|
| 1* | U95626 | chemokine, ccr-05, mip-1-alpha, mip-1-beta and |
| | | rantes |
| 2* | U03905 | chemokin, ccr-02 |
| 3 | X02956 | IFN-1, IFNa-05 |

TABLE 8c
Cytokines and Chemokines and Receptors
Upregulated at 24 Hours of CpG Exposure

| Rank | Accession # | Gene name |
|---|---|---|
| 1* | AB000887 | chemokine, ccl-19, ELC |
| 2* | L31584 | chemokine, ccr-07, EBI-1 |
| 3 | U31628 | cytokine, il-15RA |
| | L08177 | chemokine, EBI2, ebv-induced g protein-coupled |
| | | receptor 2 |

TABLE 8d
Cytokines and Chemokines and Receptors
Downregulated at 24 Hours of CpG Exposure

| Rank | Accession # | Gene name |
|---|---|---|
| 1* | U20350 | chemokine, CX3CR-01 |
| 2 | D50683 | cytokine, TGFBR2, tgf-beta receptor type ii |
| | | precursor |
| 3 | U43672 | cytokine, IL-18R1, Interleukin 18 receptor 1, |
| 4 | U58917 | cytokine, IL-17R |

Although not intending to be bound by any particular theory, it is postulated that the time course described in the Examples may roughly approximate the expression within the ppDC population in vivo during an injury, infection or disease. More specifically, the 2 hour unstimulated time point (and its corresponding marker data) may be indicative of a ppDC in vivo in a subject not having an injury, infection or disease. The 2 hour CpG stimulation time point may be characteristic of a ppDC in vivo in a subject beginning to undergo an injury, infection such as a microbial infection, or a disease such as an autoimmune disease, or other form of inappropriate immune response. The 8 hour CpG stimulation time point may be characteristic of a ppDC in vivo in a subject close to the time and place of antigen uptake and processing at the site of injury, infection or disease. The 24 hour CpG stimulation time point may be characteristic of a ppDC during the time of antigen presentation to other immune cells such as T and B cells in a secondary lymphoid site. Accordingly, an analysis of the markers expressed and not expressed at each of these time points yields valuable information regarding what proteins are involved in each of these processes. Knowledge of what genes are expressed and not expressed at each of these times leads to the discovery of agents that can be administered at these different functional stages in vivo in order to potentiate or attenuate the ongoing response. For example, expression of a chemokine receptor at the 24 hour CpG stimulation time point can indicate that such cells are receptive and responsive to the respective chemokine at that time. Accordingly, administration of that ligand at a particular time post injury (or post active infection in the case of vaccination) and/or at a particular location in a subject (i.e., a secondary lymphoid organ such as the spleen or lymph nodes) may potentiate antigen presentation by such cells.

The invention also provides methods for determining the effects of particular agents on ppDC as determined by a change in expression pattern following treatment with the agent. Thus, according to one aspect of the invention, a method is provided for identifying a candidate agent useful in the modulation of an immune response, preferably an immune response that employs a plasmacytoid dendritic cell. The method involves determining expression of a plurality (i.e., more than one) of nucleic acid molecules in a plasmacytoid dendritic cell or cell population under conditions which, in the absence of a candidate agent, permit a first amount of expression of the set of nucleic acid molecules, wherein the set of nucleic acid molecules comprises at least one nucleic acid molecule selected from the group consisting of at least a subset markers of the databases of the invention, contacting the ppDC with the candidate agent, and determining expression of a plurality of nucleic acid molecules following contact with the agent, wherein an increase in expression in the presence of the candidate agent relative to the expression in the absence of the candidate agent indicates that the candidate agent is an immune modulating agent.

Depending upon the embodiment, the immune modulating agent may be capable of inducing an immune response selected from the group consisting of a natural killer cell activity, a Th1 immune response, a Th2 immune response, and a plasmacytoid dendritic cell activity. In certain embodiments, the nucleic acid molecules comprise at least one nucleic acid corresponding to a marker having a rank of greater than 5, greater than 10, or more. In other embodiments, the plurality of nucleic acid molecules comprises at least one of each of the foregoing nucleic acid molecules. In some embodiments, the plurality of nucleic acid molecules comprises at least two, at least three, at least four, at least five nucleic acid molecules, at least 10 nucleic acid molecules, at least 20 nucleic acid molecules, at least 50 nucleic acid molecules, or even at least 100 nucleic acid molecules. In a related aspect of the foregoing aspect of the invention, the method involves determining the expression of a single nucleic acid molecule rather than a plurality. In this latter aspect, the single nucleic acid molecule is a nucleic acid molecule that is capable of uniquely characterizing an immune response.

The information provided herein, particularly with respect to co-stimulatory molecules (OX-40 and 4-1BB ligand) and negative regulatory molecules (e.g., ILT3 and IL-10 receptor) provide methods for modulating immune responses either by contacting cells with agents that trigger these markers, or by administering antisense nucleic acids that block the translation of these markers, as the desired therapeutic effect may be.

Similarly, although these markers have been identified following artificial CpG immunostimulation, the findings are applicable to the modulation of any immune response that involves pDC. Accordingly, the information provided herein provides insight as to methods for modulating inappropriate immune responses such as autoimmune responses, or uncontrolled and thus detrimental immune responses, such as those to RSV. Other agents have been previously reported to stimulate pDC including bacteria, viruses, anti-CD40 antibodies, and poly IC. The markers identified herein can be exploited to either enhance or control the immune responses that result from the stimulation of pDC by these compounds. The negative regulatory markers identified herein such as IL-10 receptor and ILT3 can be exploited to for example terminate the feedback loop that may occur in autoimmune diseases.

In one embodiment, the immune response can be modulated between a Th1 and a Th2 response. As an example, stimulation through the IL-10 receptor found to be expressed on pDC can be used to effect this modulation.

Apoptotic markers that are expressed and in some instances upregulated in pDC by CpG immunostimulation can be modulated by antisense administration.

The invention further provides screening methods for identifying agents that modulate immune responses whether the immune response is artificially induced (e.g., in a clinical setting with CpG immunostimulatory nucleic acids) or whether it is the result of an infection or an autoimmune disease, for example. The activity of ppDC may be determined in a number of ways including expression analysis, and in vitro or in vivo function. As an example of expression analysis, the expression of one, two, five, ten, or more nucleic acid molecules can be determined following treatment with the agent. The nucleic acid molecules preferably are selected from the markers listed in the databases provided herein. In important embodiments, the nucleic acid molecules are those corresponding to a particular immune activity. As an example, it was discovered according to the invention that ppDC, which heretofore have been thought capable solely of dendritic cell activity (i.e., antigen uptake, processing to presentation to other immune cells such as T cells and B cells), express natural killer (NK) cell markers, and more specifically NK activation markers, as well as Fc receptors for binding antibody. In addition, these cells were found to express lytic enzymes associated with NK activity. Accordingly, it was discovered that ppDC are capable of NK activity such as target cell lysis. This finding indicates that ppDC can be induced to kill target cells provided that they are exposed to the correct stimulus. The screening methods of the invention allow for the identification of such agents, and many such agents may be identified using the data provided herein.

Once identified, these agents may be used to induce an NK response in the ppDC in vivo or in vitro, depending upon the particular use. When used in vivo, the agent may be administered to the subject in need of NK activity systemically or in some preferred embodiments locally at the site of injury, infection or disease. The agent may be administered in combination with other agent(s) that act as chemoattractants for ppDC in order to optimize their migration to the affected site in the body.

It is to be understood that any and all of the screening methods provided herein are equally applicable to subsets of cells within the purified ppDC population described herein. As an example, subsets of ppDC can be derived by separating cells based on the expression or lack of expression of particular markers, as determined using the databases of the invention. In this way, subsets such as immature and mature ppDC can be derived and individually tested for their response to the agents and other stimuli which can be readily tested using the screening methods provided herein.

Other screening methods are also provided by the invention. In another aspect, the invention provides a screening assay for comparing the ability of other immunostimulatory nucleic acids to induce expression patterns similar to or distinct from those induced by CpG immunostimulatory nucleic acid molecules, as indicated in the databases of the invention. Analysis of immunostimulatory agents generally has involved analysis of downstream biological activities and to this extent, many immunostimulatory nucleic acids may be characterized as having similar biological effects. The present invention allows a microscopic look at the direct effects of agents such as immunostimulatory nucleic acid molecules on ppDC populations, and allows a more detailed comparison of the effects of such immunostimulatory nucleic acid molecules relative to the effects of CpG immunostimulatory nucleic acids (such as that used in the Examples). It is to be understood that this latter method is also applicable to the testing of mimics of CpG immunostimulatory nucleic acid molecules. One of ordinary skill in the art may be able to synthesize agents that conformationally mimic CpG immunostimulatory nucleic acid molecules. Such agents or other agents which are conformationally distinct from CpG may then be tested using the screening assays of the invention for their ability to similarly mimic the transcriptional effects of CpG immunostimulatory nucleic acids on ppDC populations as detailed in the databases of the invention. Small molecule synthesis is known in the art.

In another related aspect of the foregoing, the invention also embraces synthesis and testing of compounds that mimic part of the CpG response repertoire. This latter group of compounds may mimic part of, but not necessarily all, the response induced by CpG immunostimulatory nucleic acids. The use of these compounds may be suited when it is desired to stimulate one aspect of the CpG induced response but not the entire response. As an example, the screening methods of the invention can be structured such that they are able to identify a compound that induces the same changes in expression in the ppDC population as does a 2 hour exposure to CpG immunostimulatory nucleic acid (as in Examples and in the databases) but does not induce the same changes of expression as are present following 8 hours, or in some instances 24 hours of exposure to CpG immunostimulatory nucleic acids. Conversely, it is possible to identify compounds that induce changes in expression in pDC that are similar to those observed following 8 hours or in some instances 24 hours of CpG exposure but that do not induce the same changes as those observed following 2 hours of CpG exposure.

The screening methods can be further used to determine the effects of other immunostimulatory agents as compared to the effects of CpG immunostimulatory nucleic acids. For example, with knowledge of both the biological outcome and the changes in expression patterns induced by CpG immunostimulation, it is now possible to characterize other immunostimulatory agents relative to these two parameters, and to identify and categorize such agents based on their ability to effect the entire CpG response or a portion thereof.

Yet another unexpected finding is the observation that resting state and stimulated ppDCs express 4-1BB ligand which is a T cell co-stimulatory molecule. This molecule was not previously known to be expressed by ppDC. This finding is the basis for therapeutic methods for either inducing or attenuating an immune response that involves ppDC and optionally T cells (such as the antigen presentation and recognition that occurs between these cell types in secondary lymphoid tissues).

Drug screening methods using microarrays such as those described herein are also described by Gerhold et al. (Physiol. Genomics, 5:161-170, 2001.)

It is to be further understood that the synthesis and screening methods described herein can be further applied to the identification of compounds that act as antagonists to CpG stimulation. Such compounds are useful, inter alia, as modulators of CpG response in vivo. As an example, such agents may be identified according to their ability to down-regulate the expression of particular activation markers (such as for example cytokine or chemokine receptors, or cell cycle factors), or conversely to up-regulate the expression of particular suppression or inhibitory markers (such as for example an apoptosis related marker).

As used herein, antagonists are compounds that tend to nullify the action of another, such as a drug (e.g., CpG immunostimulatory nucleic acid). As used herein, agonists are compounds that stimulate a physiological activity normally stimulated by another compound such as a drug (e.g., CpG immunostimulatory nucleic acid), and are thereby capable of triggering a biochemical response.

A CpG oligonucleotide is an oligonucleotide which includes at least one unmethylated CpG dinucleotide. An oligonucleotide containing at least one unmethylated CpG dinucleotide is a nucleic acid molecule which contains an unmethylated cytosine-guanine dinucleotide sequence (i.e. “CpG DNA” or DNA containing a 5′ cytosine followed by 3′ guanosine and linked by a phosphate bond) and activates the immune system.

Mimics of CpG immunostimulatory nucleic acids can be synthesized from nucleotides including purines and pyrimidines, or other biomolecules including but not limited to saccharides, fatty acids, sterols, isoprenoids, amino acids, derivatives or structural analogs of the above, or combinations thereof and the like. Phage display libraries and chemical combinatorial libraries can be used to develop and select synthetic compounds which are suitable candidates as CpG immunostimulatory nucleic acids mimics. Also envisioned in the invention is the use of antagonists made from peptoids, random bio-oligomers (U.S. Pat. No. 5,650,489), benzodiazepines, diversomeres such as dydantoins, benzodiazepines and dipeptides, nonpeptidal peptidomimetics with a beta-D-glucose scaffolding, oligocarbamates or peptidyl phosphonates.

Many if not all of these compounds can be synthesized using recombinant or chemical library approaches. A vast array of candidate antagonists can be generated from libraries of synthetic or natural compounds. Libraries of natural compounds in the form of bacterial, fungal, plant and animal extracts are available or can be readily produced. Natural and synthetically produced libraries and compounds can be readily modified through conventional chemical, physical, and biochemical means. CpG immunostimulatory nucleic acids may be subjected to directed or random chemical modifications such as acylation, alkylation, esterification, amidification, etc. to produce structural analogs, which may function as antagonists or agonists.

The methods of the invention utilize this library technology to identify small molecules including small peptides and small oligonucleotide analogs. These small molecules can be screened on the basis of binding to a toll-like receptor 9, which is bound by a CpG immunostimulatory nucleic acid. One advantage of using libraries for analog identification is the facile manipulation of millions of different putative candidates of small size in small reaction volumes (i.e., in synthesis and screening reactions). Another advantage of libraries is the ability to synthesize antagonists which might not otherwise be attainable using naturally occurring sources, particularly in the case of non-peptide or non-nucleotide moieties.

With knowledge of the markers that are up-regulated in response to exposure to CpG immunostimulatory nucleic acid molecules, it is possible to design therapeutic methods for modulating an immune response using antisense nucleic acid molecules specific for such markers, or expression vectors encoding such markers, or compounds which otherwise influence the expression or activity of the marker.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence. It is preferred that the antisense oligonucleotide be constructed and arranged so as to bind selectively with the target under physiological conditions, i.e., to hybridize substantially more to the target sequence than to any other sequence in the target cell under physiological conditions.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness. The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose.

The present invention, thus, contemplates pharmaceutical preparations containing modified antisense molecules that are complementary to and hybridizable with, under physiological conditions, nucleic acids corresponding to the markers of the databases provided herein in the tables, together with pharmaceutically acceptable carriers. Antisense oligonucleotides may be administered as part of a pharmaceutical composition. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a patient. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

The invention further provides in other aspects nucleic acid and peptide arrays. The nucleic acid arrays consist essentially of a subset of markers listed in the databases. This subset of markers will be different depending on what is being tested. As an example, if the array is intended for use as a screening tool for identification or confirmation of a cell designation (e.g., confirming that a cell is a ppDC), then the markers are preferably those chosen from markers that either expressed or not expressed in the 2 hour unstimulated data set. The array may a minimum of one marker and less than 12,000 markers. In preferred embodiments, the array will contain the minimum number of markers required to accurately identify a cell as either a ppDC, or a select subset of ppDC (e.g., immature ppDC or mature ppDC). The array may contain at least 2, at least 3, at least 4, at least 5, at least 6, at least 7, at least 8, at least 9, at least 10, and so on, up to an including not more than 12,000 markers. These markers can be those that are expressed by in the 2 hour unstimulated data set. Examples of markers highly expressed in unstimulated ppDC include but are not limited to IFNα-2, CXCL-11 (ITAC), IGF-1, MYD116, BFL-1, CCL-04 (MIP-1β), TNFβ, and CXCL-10 (IP-10). Examples of markers that are expressed at low levels in unstimulated ppDC include but are not limited to IFNα-1/13, CXCL-02 (MIP-1α), DNAJB4, PE24 prostaglandin E receptor, and oxytocin. Examples of markers that are expressed either at negligible levels or not at all include but are not limited to RIPKJ2, CCL-05 (RANTES), FXC1, CSRP2, and IFNβ-1. It is within the skill of the ordinary artisan to select markers suitable for the nucleic acid array.

A solid-phase nucleic acid molecule array consists essentially of a plurality of nucleic acid molecules, expression products thereof, or fragments thereof, wherein at least two and less than all of the nucleic acid molecules selected from the group of markers listed in the Tables (including expression products thereof, or fragments thereof) are fixed to a solid substrate. In some embodiments, the solid-phase array further comprises at least one control nucleic acid molecule. In certain embodiments, the plurality of nucleic acid molecules comprises at least three, at least four, or even at least five nucleic acid molecules. In preferred embodiments, the set of nucleic acid molecules comprises a maximum number of 100 different nucleic acid molecules. In important embodiments, the set of nucleic acid molecules comprises a maximum number of 10 different nucleic acid molecules. In even more important embodiments, the set of nucleic acid molecules comprises a maximum number of 5 different nucleic acid molecules.

According to the invention, standard hybridization techniques of microarray technology are utilized to assess patterns of nucleic acid expression and identify nucleic acid expression. Microarray technology, which is also known by other names including DNA chip technology, gene chip technology, and solid-phase nucleic acid array technology, is well known to those of ordinary skill in the art and is based on, but not limited to, obtaining an array of identified nucleic acid probes on a fixed substrate, labeling target molecules with reporter molecules (e.g., radioactive, chemiluminescent, or fluorescent tags such as fluorescein, Cy3-dUTP, or Cy5-dUTP), hybridizing target nucleic acids to the probes, and evaluating target-probe hybridization. A probe with a nucleic acid sequence that perfectly matches the target sequence will, in general, result in detection of a stronger reporter-molecule signal than will probes with less perfect matches. Many components and techniques utilized in nucleic acid microarray technology are presented in The Chipping Forecast, Nature Genetics, Vol.21, January 1999, the entire contents of which is incorporated by reference herein.

According to the present invention, microarray substrates may include but are not limited to glass, silica, aluminosilicates, borosilicates, metal oxides such as alumina and nickel oxide, various clays, nitrocellulose, or nylon. In all embodiments a glass substrate is preferred. In some embodiments, the nucleic acid molecules are fixed to the solid substrate by covalent bonding. According to the invention, probes are selected from the group of nucleic acids including, but not limited to: DNA, genomic DNA, cDNA, and oligonucleotides; and may be natural or synthetic. Oligonucleotide probes preferably are 20 to 25-mer oligonucleotides and DNA/cDNA probes preferably are 500 to 5000 bases in length, although other lengths may be used. Appropriate probe length may be determined by one of ordinary skill in the art by following art-known procedures. Probes may be purified to remove contaminants using standard methods known to those of ordinary skill in the art such as gel filtration or precipitation. Preferably the nucleic acids fixed to the solid support are or comprise unique fragments as described herein.

In one embodiment, the microarray substrate may be coated with a compound to enhance synthesis of the probe on the substrate. Such compounds include, but are not limited to, oligoethylene glycols. In another embodiment, coupling agents or groups on the substrate can be used to covalently link the first nucleotide or oligonucleotide to the substrate. These agents or groups may include, but are not limited to: amino, hydroxy, bromo, and carboxy groups. These reactive groups are preferably attached to the substrate through a hydrocarbyl radical such as an alkylene or phenylene divalent radical, one valence position occupied by the chain bonding and the remaining attached to the reactive groups. These hydrocarbyl groups may contain up to about ten carbon atoms, preferably up to about six carbon atoms. Alkylene radicals are usually preferred containing two to four carbon atoms in the principal chain. These and additional details of the process are disclosed, for example, in U.S. Pat. No. 4,458,066, which is incorporated by reference in its entirety.

In one embodiment, probes are synthesized directly on the substrate in a predetermined grid pattern using methods such as light-directed chemical synthesis, photochemical deprotection, or delivery of nucleotide precursors to the substrate and subsequent probe production.

In another embodiment, the substrate may be coated with a compound to enhance binding of the probe to the substrate. Such compounds include, but are not limited to: polylysine, amino silanes, amino-reactive silanes (Chipping Forecast, 1999) or chromium (Gwynne and Page, 2000). In this embodiment, presynthesized probes are applied to the substrate in a precise, predetermined volume and grid pattern, utilizing a computer-controlled robot to apply probe to the substrate in a contact-printing manner or in a non-contact manner such as ink jet or piezo-electric delivery. Probes may be covalently linked to the substrate with methods that include, but are not limited to, UV-irradiation. In another embodiment probes are linked to the substrate with heat.

Nucleic acids that can be applied to the array are selected from the group, including but not limited to: DNA, genomic DNA, cDNA, RNA, mRNA and may be natural or synthetic. In all embodiments, nucleic acid molecules from subjects undergoing or requiring an immune response, are preferred. In certain embodiments of the invention, one or more control nucleic acid molecules are attached to the substrate. Preferably, control nucleic acid molecules allow determination of factors including but not limited to: nucleic acid quality and binding characteristics; reagent quality and effectiveness; hybridization success; and analysis thresholds and success. Control nucleic acids may include, but are not limited to, expression products of genes such as housekeeping genes or fragments thereof.

To select a set of immune response associated markers, the expression data generated by, for example, microarray analysis of gene expression, is preferably analyzed to determine which genes are significantly differentially expressed in response to stimulation with the CpG immunostimulatory nucleic acid at the 2 hour, 8 hour, or 24 hour time point. The significance of gene expression can be determined using Permax computer software, although any standard statistical package that can discriminate significant differences is expression may be used. Permax performs permutation 2-sample t-tests on large arrays of data. For high dimensional vectors of observations, the Permax software computes t-statistics for each attribute, and assesses significance using the permutation distribution of the maximum and minimum overall attributes. The main uses include determining the attributes (genes) that are the most different between stimulated and unstimulated samples, or in other embodiments between different subsets of cells (e.g., immature versus mature ppDC subsets; confirmed ppDC and putative ppDC, and the like), or in yet other embodiments, between different patients, measuring “most different” using the value of the t-statistics, and their significance levels.

The use of any of the foregoing microarray methods to determine expression of immune response associated markers can be done with routine methods known to those of ordinary skill in the art and the expression determined by protein measurement methods may be correlated to predetermined levels of a marker used as a prognostic method for selecting treatment strategies for patients in need of or having an immune response.

The invention also provides peptide arrays. The peptide arrays provided for herein can comprise either binding partners of the peptides or polypeptides encoded by the markers listed in the databases provided herein, or alternatively, can comprise fragments (preferably unique fragments) of the polypeptides or peptides encoded by the markers. In the first variation, the peptide array could commonly comprise antibodies or antibody fragments that bind specifically to peptides or polypeptides encoded by the markers listed in the databases of the invention. Such an array would be useful in determining the level of protein expression from these markers. Additionally, this array is useful as another way of fingerprinting the ppDC. The advantage of using a peptide array over a nucleic acid array in some instances is the ability to harvest larger amounts of peptides and polypeptides from cells as compared to mRNA. The peptide array analysis can be used alongside of or in place of the nucleic acid array in the methods described herein.

The peptide array can also comprise peptides, polypeptides or fragments thereof expressed by the ppDC. Such a peptide array can be used for screening compounds that bind to protein expression products in the ppDC. In this way, the peptide array can be used as a simple binding screen for compounds that bind to peptides or polypeptides expressed by the ppDC. Compounds so identified can be further worked-up in order to test their effect on ppDC, ppDC function and ppDC expression patterns, as described herein. Only with the knowledge derived from the databases of the invention can a peptide array be uniquely designed so that it captures all, or a subset of, proteins that are expressed by ppDC.

In these latter aspects of the invention, standard techniques of microarray technology are utilized to assess expression of polypeptides and/or identify compounds that bind such polypeptides. The compounds that bind such polypeptides can be small molecule compounds such as those described whose synthesis is described herein. In other embodiments, these “compounds” may be constituents of a cell (in some instances, preferably a ppDC). In still other embodiments, the “compounds” may be cells themselves such as immune cells (e.g., T and B cells) and the like.

Protein microarray technology, which is also known by other names including protein chip technology and solid-phase protein array technology, is well known to those of ordinary skill in the art and is based on, but not limited to, obtaining an array of identified peptides or proteins on a fixed substrate, binding target molecules or biological constituents to the peptides, and evaluating such binding. See, e.g., G. MacBeath and S. L. Schreiber, “Printing Proteins as Microarrays for High-Throughput Function Determination,” Science 289(5485):1760-1763, 2000.

In some important embodiments, antibodies or antigen binding fragments thereof that specifically bind polypeptides selected from the group encoded by markers listed in the databases are attached to the microarray substrate in accordance with standard attachment methods known in the art. These arrays can be used to quantify the expression of the polypeptides identified herein.

In some embodiments of the invention, one or more control peptide or protein molecules are attached to the substrate. Preferably, control peptide or protein molecules allow determination of factors such as peptide or protein quality and binding characteristics, reagent quality and effectiveness, hybridization success, and analysis thresholds and success.

The agents for use in some of the methods of the invention as well as in some of the peptide arrays include antibodies and antibody fragments.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes use of so-called single chain antibodies.

In many aspects of the invention, including the screening assays, the identification of agents that mimic, enhance or antagonize the effects of CpG immunostimulatory nucleic acids, and binding to nucleic acid or peptide arrays, employ binding of one component to another. Standard binding assays and the conditions of such assays are well known in the art. The nature of the assay is not essential provided it is sufficiently sensitive to detect binding of a small number of interactions, such as those between a small molecule compound and the toll-like receptor 9, or those between a cDNA from a sample and an nucleic acid on a nucleic acid array, or a peptide from a sample and a peptide array.

As mentioned above, the invention provides a number of applications for the databases of the invention. Included in these methods are screening, categorizing and monitoring methods that employ the data of the database.

In one aspect, the invention allows for subjects to be screened and potentially characterized according to their ability to respond to an immunostimulatory nucleic acid such as CpG immunostimulatory nucleic acid. Such subjects may be possible candidates for treatment with CpG immunostimulatory nucleic acid, or alternatively, they may be in the process of receiving CpG immunostimulatory nucleic acid and it is of interest to determine whether their ppDC are continuing to respond to such treatment (i.e., ascertaining whether ppDC have become unresponsive to the treatment). Alternatively, such subjects may be undergoing treatment and it is of interest to determine the efficacy of the treatment. Efficacy of treatment can be determined by testing for the presence of ppDC (especially those having a particular transcript expression profile) in the subject at a certain time or at a certain location in the subject following treatment. Moreover, it is possible to diagnose a disorder, and in particular a stage of the disorder, based on which ppDC are present in the subject (and in some instances, preferably at the site of a lesion such as a cancerous lesion) and what markers are expressed by such ppDC. Expression profiles corresponding to any of the foregoing can be compared to the expression profiles contained within the databases, or some subset of such profile. It has been reported that dendritic cells are reduced in number in the circulation of cancer patients with metastases. (Lissoni et al., J. Biol. Regul. Homeost. Agents, 1999, 13(4):216-9.) Accordingly, a similar analysis of ppDC in subjects having a disorder such as cancer, an infectious disease, an allergy, or asthma, can be carried out, by analyzing the transcripts expressed in ppDC harvested from such subjects and comparing those expression patterns with that of the databases provided herein. This type of analysis can provide more fine-tuned, detailed characterization of a disease over and above the classical histological and scant genetic characterizations that are currently available. Additionally, this type of analysis can be used to flag subjects for less aggressive, more aggressive, and generally more tailored therapy to treat the disorder.

The screening methods described herein for identifying agents that mimic CpG immunostimulatory nucleic acids, can also be used for validating the efficacy of agents. Agents of either known or unknown identity can be analyzed for their effects on gene expression in ppDC using methods such as those described herein. Briefly, purified populations of ppDC or subsets thereof (based on further purification on the basis of a subset of markers from the databases) are exposed to the agent, preferably in an in vitro culture setting, and after set periods of time, the entire cell population or a fraction thereof is removed and mRNA is harvested therefrom. Either mRNA or cDNA is then applied to a nucleic acid array such as that used in the Examples or in some embodiments a nucleic acid array that consists of a subset of those markers. Hybridization readouts are then compared to the data of provided herein and conclusions are drawn with respect to the similarity of the action of the agent to that of CpG immunostimulatory nucleic acid. These methods can be used for identifying novel agents, including nucleic acid and nucleic acid analog based agents, as well as confirming the identity of agents that are suspected of being immunostimulatory agents.

In yet another aspect, the invention provides a method for manufacturing a factor such as but not limited to a chemokine or cytokine. The databases indicate that a number of factors are produced by ppDC either prior to or following stimulation with CpG immunostimulatory nucleic acids for various times. As an example, the 2 hour stimulated data set indicates that IFNα types, CXCL-11 (ITAC), CXCL-09 (Mig), CCL-05 (RANTES), CCL-04 (MIP-1β), TNFβ, CXCL-10 (IP-10), IL-6, 4-1BB ligand, CCL-03 (MIP-1α), CXCL-02 (MIP-1α), IL-1β, oxytocin, CCL-19 (ELC), NK4, vasopressin-neurophysin 2-copeptin, LSP-1, CXCL-01, CXCL-03 (GROγ), CXCL-08 (IL-8), CCL-18, and the like. Generally, the method can be used to produce or manufacture large quantities of a particular factor (or a combination of factors). Factors can be produced from either unstimulated or stimulated ppDC depending upon where the maximum factor production is predicted to occur. One of ordinary skill in the art is capable of determining the maximum production of a given factor based on the information provided herein. The sample from which to derive factor can also depend upon what other factors are produced in that sample. For example, it may be desirable in some instances to chose a sample, treatment and time point at which there are few other factors produced in order to simply factor purification following harvest of a culture supernatant. Selection of a factor and the proper stimulation and time point will depend upon analysis of its rank, among other things. If the factor is produced following stimulation with CpG immunostimulatory nucleic acids, then it will be important to select markers that are not only induced following CpG immunostimulation but which also express high levels of the factor. Harvest and purification of factors can be performed using procedures that are routine in the art. Such procedures may employ binding partners that are fixed onto a solid state such as a dish or a column. The binding partners can be antibodies or fragments thereof but they are not so limited.

One of the surprising findings of the information provided by the invention is the observation that anti-apoptotic factors are induced following CpG immunostimulation. An example of such an anti-apoptotic factor is BFL-1 (at the 2 hour stimulation time point). The finding that CpG immunostimulatory nucleic acids upregulate expression of anti-apoptotic factors suggest that CpG functions as a chemoprotectant. As used herein, a chemoprotectant is an agent that protects cells from programmed cell death, either directly, or more importantly via the induction of anti-apoptotic factors within a cell such as a ppDC. This leads to methods for identifying other agents that similar to CpG can induce anti-apoptotic factors. Accordingly, the induction of anti-apoptotic factors can be used as a readout for identifying further chemoprotectants, and agents so identified can be compared to the induction by CpG immunostimulatory nucleic acids in order to assess the relative efficacy of the agent for this function.

Another unexpected finding according to the invention is that unstimulated and to greater extents stimulated ppDC express (i.e., CXCL-10). This factor is known to have anti-angiogenic activity (with corresponding anti-tumour activity, given its ability to inhibit angiogenesis at a tumour). This finding leads to methods for stimulating ppDC using CpG immunostimulatory nucleic acids and/or mimics thereof at sites where angiogenesis is undesirable (such as tumours, placenta mass, etc.) in vivo in order to increase in vivo production of IP-10. IP-10 is also a suitable candidate to be produced in vitro using the methods described above.

In still other aspects of the invention, methods are provided for potentiated immune responses in vivo (and in some instances in vitro or ex vivo) by capitalizing on the markers expressed by ppDC either in a stimulated or unstimulated form. As an example, knowing what markers are expressed by ppDC in the absence of CpG immunostimulation allows one to tailor a cocktail for stimulating ppDC either with or without CpG immunostimulatory nucleic acids. Knowing what markers are expressed by ppDC at various times during CpG immunostimulation allows one to tailor a cocktail for further stimulating (or potentiating) the immune response derived from CpG immunostimulatory nucleic acids. As an example, knowledge that ppDC express a particular chemokine receptor as a result of CpG immunostimulation indicates that exposure of such cells to the respective chemokine at that time (or some time thereafter) can be useful in increasing the immune response.

In one embodiment, the immune response that is potentiated is an innate immune response, and in another embodiment, the immune response that is potentiated is an adaptive immune response. In one embodiment, agents are screened for the ability to upregulate CD40.

The invention intends to embrace in yet another aspect molecules that bind to either a ppDC and thereby induce an immune response that is approximately identical to the immune response induced by CpG immunostimulatory nucleic acids. A subset of such molecules will bind to TLR9 (toll like receptor 9), as do some CpG immunostimulatory nucleic acids. Accordingly, the invention also embraces molecules that bind to TLR9 positive cells and thereby induce a response is such cells that resembles the effects of CpG immunostimulatory nucleic acid on the ppDC of the present invention. The response in this latter aspect can be defined in terms of the induction or reduction of expression of one or more markers, relative to their expression in the databases.

Knowledge of chemokine receptors expressed by ppDC either in the resting state or following CpG immunostimulation allows one to potentiate an immune response in vivo by administration of chemokines to those receptors. Administration of such chemokines can induce the migration of ppDC to the site of injury, infection or disease. The following table indicates those chemokine receptors that are expressed in ppDC either in an unstimulated form or following CpG immunostimulation. The table lists chemokine receptors having high levels of expression.

TABLE 9

| | 2 hour | | |
|---|---|---|---|
| Unstimulated* | stimulation | 8 hour stimulation | 24 hour stimulation |
| CCR-07 | CCR-07 | | CCR-07 |
| CXCR-03 | | | CXCR-03 |
| CCR-02 | | | |
| C3XCR-01 | | | |
| CXCR-04 | | CXCR-04 | |
| | | | CXCR-05 |

*may be expressed at either 2 hours, 8 hours, or 24 hours of culture in the absence of CpG immunostimulation.

The invention provides further information regarding other growth factor receptors, and cytokine receptors expressed by ppDC in either a resting state or following CpG immunostimulation. To the extent that the ligands for such receptors are known, it is possible to design therapeutic methods for stimulating these dendritic cells via the administration of known ligands to the now-known expressed receptors. Additionally, it should be possible to target such cells with ligands that are known to inhibit cells perhaps via the same receptor or via different molecules that are also expressed by these cells.

Knowledge of markers that are down-regulated in response to CpG immunostimulation provides information regarding which markers can be effectively targeted in a combination therapy of CpG immunostimulation and antibody-dependent cell cytotoxicity (ADCC). ADCC is used to kill target cells expressing the marker-to which the administered antibody has specificity. An understanding of what markers are upregulated as well as those that are down regulated as a result of CpG immunostimulation allows an ADCC protocol to be tailored in combination with CpG immunostimulation. As an example, markers that are downregulated are generally not good candidates for ADCC. In some instances it is possible that although CpG immunostimulation vastly reduces the level of expression of the marker relative to the unstimulated sample, the marker may still be expressed at relatively high levels (and thus would still be an effective target of ADCC). Conversely, markers that are upregulated in response to CpG immunostimulation can be suitable targets for ADCC. As with down-regulated markers, the marker may be upregulated in response to CpG immunostimulation, yet the ultimate absolute level of expression may still be too low to effectively be useful as a target of ADCC. The specific finding that Fc IgG receptor is downregulated in response to CpG immunostimulation (e.g., at the 24 hour time point) is especially useful for designing ADCC strategies.

Another surprising discovery upon which the invention is based is the finding that CpG immunostimulation leads to an increase in the expression level of COX-2, a prostaglandin-endoperoxide synthase known to be a target of aspirin. Following only 2 hours of culture with CpG immunostimulatory nucleic acids, COX-2 is upregulated approximately 60 fold. This finding leads to methods for potentiating the effects of aspirin by prior or simultaneous administration of aspirin, and indicates the therapeutic utility of CpG immunostimulation for medical indications calling for treatment with aspirin. Examples of such indications include headaches and cardiovascular disorders.

To the extent that ppDC are also involved in aberrant processes in the body, the information provided herein also allows for the design of therapeutic or prophylactic strategies for targeting these cells. For example, it has been reported that ppDC are involved in rhinitis and arthritis. Accordingly, it is may be desirable eliminate such cells during the development of these disorders. One way of targeting these cells would be to determine a marker or set of markers that could be targeted using ADCC, as described elsewhere herein. Another way of targeting these cells is to take advantage of the apoptotic factors which have been demonstrated to be expressed in these cells according to the invention. Other methods for targeting such cells are well within the realm of the ordinary artisan.

In a related aspect, the invention embraces the methods for modulating inflammation and inflammatory processes. For example, the cells can be used to screen for compounds that upregulate (i.e., agonists) or downregulate (i.e., antagonists) inflammatory markers. These methods can be performed in the absence of CpG immunostimulation by developing agonists and antagonists that modulate the resting state expression profile of these cells (i.e., the 2 hour unstimulated data set). Another unexpected finding of the present invention is the observation that proinflammatory chemokines are expressed (and in some instances at high levels) at 2 hours. Accordingly, the cells can be used to screen for agents that attenuate or inhibit the expression of such pro-inflammatory chemokines.

It was further discovered according to the invention that ppDC express chemokines that attract T cells following 24 hours of CpG immunostimulation. This finding can be the basis for a therapeutic strategy for modulating the immune response, by for example attenuating such chemokine release, or alternatively, enhancing said release.

The expression fingerprints provided herein can also be used as global indicators of dendritic cell stimulation, maturation and immune response efficacy. Dendritic cells grown in vitro, or harvested in a temporal or spatial manner from a subject can be analyzed according to this expression fingerprint in order to more fully characterize the dendritic cell and to determine its potential for immune response involvement, or its past immune response involvement.

The expression fingerprint of ppDC provided herein allows one of ordinary skill to determine that set of signalling molecules that are expressed in such cells, thereby allowing a determination of what signaling pathways are activated (and which are not activated) in these cells. Accordingly, if it is desirable to stimulate such cells further, then the cells can be contacted with agents that stimulate a specific pathway known to be active. If on the other hand it is desirable to inhibit the stimulation of such cells, then the cells can be contacted with an agent(s) known to inhibit the same pathway. The intimate knowledge of what transcripts are expressed and which are not expressed in these cells allows for tailoring of therapies both to the cells, and in some embodiments to particular subjects (once their ppDC expression profile is known).

It was also discovered according to the invention that TNFβ is produced in response to CpG immunostimulation by these cells. Using prior art methods including ELISA it has not been previously possible to determine the production of this factor in response to CpG immunostimulation. Now, in accordance with the invention, it has been discovered that not only is TNFβ expressed in such cells following CpG immunostimulation, but the level of expression is higher than that of TNFα. This finding leads to screening methods for identifying agents that are agonists and those that are antagonists to the production of this factor following CpG immunostimulation. In addition, this finding leads to therapeutic methods for treating subjects undergoing CpG immunostimulation to either enhance or downmodulate this effect. In yet another aspect, this finding leads to therapeutic methods for treating conditions that benefit from the administration of TNFβ by administering CpG immunostimulatory nucleic acids in place or in combination with TNFβ or other agents that induce TNFβ production.

Many of the illustrative embodiments provided herein refer to in vivo uses for agents identified according to the screening methods of the invention. However, it is to be understood that these are intended for illustrative purpose only, and the invention embraces the use of such agents in the stimulation and/or attenuation of dendritic cells either in vivo or ex vivo. Dendritic cells stimulated ex vivo using the agents identified in the screening methods described herein can then be used in therapeutic regimens for treating a variety of disorders, and particularly antigen specific disorders.

The use of dendritic cells in vaccines and in antigen specific therapies (such as anti-tumour therapies that involve priming of immune cells with antigen ex vivo prior to re-introduction into a subject). The dendritic cells of the invention are suitable for use in these methods, and it is one aim of the invention to identify agents that modulate the activity of dendritic cell ex vivo as well as in vivo.

In some subjects, particularly those having or at risk of developing a disorder which is responsive to dendritic cell therapy, it is possible to stimulate ppDC in vivo in order to potentiate antigen specific immune responses, including antigen recognition and uptake by ppDC, and antigen presentation by ppDC to other immune cells such as T and B cells. Subjects that are possible candidates for such treatment can be initially screened for the ability of their ppDC to respond to such treatment.

With the advent of single cell gene expression analysis, it should be possible to analyze the expression profiles within individual subjects, and in some instances in individual ppDC, in response to CpG immunostimulation or in response to other confirmed or putative immunostimulants. The expression within single cells or within low numbers of cells is facilitated by amplifying mRNA transcripts in such cells using a 3′ amplification approach, as described by Billia et al. (Blood. April. 15, 2001;97(8):2257-68) and Brady et al. (Methods Enzymol. 1993;225:611-23). Combining this approach with the microarray technology described herein, the individual expression patterns (i.e., fingerprints for a particular individual's ppDC) can be determined and used to tailor a personalize therapeutic regimen for that subject.

In yet another aspect of the invention, the information provided in the databases of the invention can be used to stimulate proliferation of ppDC in vitro where it is desirable to increase numbers of DC for example prior to reintroduction into a subject. In a similar fashion, proliferation of ppDC can be effected in vivo by administering agents that stimulate proliferation, based on the data provided herein.

In a related aspect, it is possible to stimulate further development of ppDC along the NK lineage, now that it has been discovered that ppDC express certain markers heretofore considered NK specific.

In a general sense, the information provided herein can be exploited directly provided there are binding partners (either agonists or antagonists) known for the various markers. Accordingly, while many of the illustrative embodiments described herein are directed towards the use of newly discovered agents (discovered using the screening assays described herein), it is to be understood that such methods are also intended to employ already existing ligands and binding partners.

In a further embodiment, the invention provides a method for wound healing (i.e., the treatment of wounds). The method involves administering to a subject in need of such treatment an agent that recapitulates the complete immune response induced by CpG immunostimulatory nucleic acid molecule. This agent would be selected and identified based on the screening methods provided herein. As used herein a wound is a lesion in the body that is associated with and is preferably the site of a disorder. Accordingly, a wound can be a tumour, or a site of infection, but is not so limited. In a related embodiment, the ppDC can be stimulated in vitro and then injected in a subject. The cells and the agents can be administered either locally or systemically. In related embodiments, it is possible to treat a wound via the sequential administration of agents that perform portions of the immune response induced by CpG immunostimulatory nucleic acids. For example, in one aspect, a first agent is administered in order to recruit ppDC to the wound, following which a second agent is administered (at a set time after) to induce the migration of ppDC to secondary lymphoid organs such as the lymph nodes. In one embodiment, a number of other agents can be administered that act in between the first and second agent or that function prior to the first agent or following the second agent. In one embodiment, a third agent is administered that is capable of enhancing the memory of the immune system to the particular presented antigen.

In a more specialized application, the ppDC of the invention can be administered along with an antigen vaccine in order to potentiate the immune response to the vaccine antigen. Preferably the ppDC are stimulated either in vitro or in vivo in order to enhance their antigen uptake and presentation functions for example.

The invention therefore embraces a wide variety of methods for modulating or engineering an immune response by allowing for the identification of agents that enhance or attenuate particular defined aspects of an immune response. Preferably, segments of the immune response are defined according to expression pattern for one or more markers selected from the group of markers listed in the tables provided herein.

In yet another aspect, the invention embraces the genus of molecules that induce stimulation of ppDC similar to that induce by CpG immunostimulatory nucleic acids. These compounds are preferably comprised of nucleotides or nucleotide analogs and are even more preferably oligonucleotides. These compounds also comprise at least one C (or a structural analog thereof) and at least one G (or a structural analog thereof), although it is not necessary that the C and G be contiguous to each other. Importantly, these compounds must be capable of binding to the TLR9. Accordingly, they must structurally mimic CpG immunostimulatory nucleic acids, and more importantly they must mimic the conformation of CpG immunostimulatory nucleic acids when in contact with TLR9. Co-crystallization of TLR9 with CpG immunostimulatory nucleic acids can be used to determine the particular structure adopted by the nucleic acid in order for it to bind and stimulate the TLR9. This information can in turn be applied to the selection of agents that induce ppDC stimulation.

It is to be understood that markers provided herein can be used for various purposes and the applications and selection of markers for these applications will depend upon whether expressed or differentially expressed are more important.

“Expression,” as used herein, refers to nucleic acid (i.e., mRNA) expression.

As used herein, a subject is a mammal or a non-human mammal. In all embodiments human nucleic acids, polypeptides, and human subjects are preferred.

As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulated by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulated by standard techniques known to those of ordinary skill in the art.

Unique fragment of the markers provided herein can be used in a number of aspects of the invention. Unique fragments can be determined using different methodologies. A “unique fragment,” as used herein with respect to a nucleic acid is one that is a ‘signature’ for the larger nucleic acid.

“Agents that increase expression” of a nucleic acid, as used herein, are known in the art, and refer to sense nucleic acids, polypeptides encoded by the nucleic acids, and other agents that enhance expression of such molecules (e.g., transcription factors specific for the nucleic acids that enhance their expression). Any agents that increase expression of a molecule (and as described herein, increase its activity), are useful according to the invention.

The methods of the invention are useful in both the acute and the prophylactic treatment of any of the foregoing conditions. As used herein, an acute treatment refers to the treatment of subjects having a particular condition. Prophylactic treatment refers to the treatment of subjects at risk of having the condition, but not presently having or experiencing the symptoms of the condition.

In its broadest sense, the terms “treatment” or “to treat” refer to both acute and prophylactic treatments. If the subject in need of treatment is experiencing a condition (or has or is having a particular condition), then treating the condition refers to ameliorating, reducing or eliminating the condition or one or more symptoms arising from the condition. In some preferred embodiments, treating the condition refers to ameliorating, reducing or eliminating a specific symptom or a specific subset of symptoms associated with the condition. If the subject in need of treatment is one who is at risk of having a condition, then treating the subject refers to reducing the risk of the subject having the condition.

The invention provides pharmaceutical preparations of the agents of the invention. These pharmaceutical preparations comprise the agent of the invention and also a pharmaceutically acceptable carrier. The pharmaceutical preparations may be administered in effective amounts. The effective amount will depend upon the mode of administration, the particular condition being treated and the desired outcome. It will also depend upon, as discussed above, the stage of the condition, the age and physical condition of the subject, the nature of concurrent therapy, if any, and like factors well known to the medical practitioner. For prophylactic applications, it is that amount sufficient to delay the onset of, inhibit the progression of, or halt altogether the particular condition being treated, thereby producing patient benefit. For therapeutic applications, it is that amount sufficient to achieve a medically desirable result, thereby producing patient benefit. In some instances, patient benefit may be measured by a reduction in morbidity and/or mortality. In some cases this is a decrease in cell maturation and/or proliferation, or an increase in either of these two parameters.

Generally, doses of active compounds of the present invention would be from about 0.01 mg/kg per day to 1000 mg/kg per day. It is expected that daily doses ranging from 1-500 mg/kg, and preferably doses ranging from 1-100 mg/kg, and even more preferably doses ranging from 0.001-50 mg/kg, and most preferably doses ranging from 0.001-10 mg/kg will be suitable. A variety of administration routes are available. The methods of the invention, generally speaking, may be practiced using any mode of administration that is medically acceptable, meaning any enteral or parenteral mode that produces effective levels of the active compounds without causing clinically unacceptable adverse effects. Such modes of administration include oral, rectal, topical, nasal, intrapulmonary, intracavitary, transdermal, interdermal, transmucosai, subcutaneous, intravenous, intraarterial, intramuscular, or local routes. The term “parenteral” includes subcutaneous, intravenous, intramuscular, or infusion. Injectable routes such as intravenous or intramuscular routes are not particularly suitable for long-term therapy and prophylaxis. They could, however, be preferred in emergency situations. Oral administration will be preferred for prophylactic or therapeutic treatment because of the convenience to the patient as well as the dosing schedule.

When peptides are used therapeutically, in certain embodiments a desirable route of administration is by pulmonary aerosol. Techniques for preparing aerosol delivery systems containing peptides are well known to those of skill in the art. Generally, such systems should utilize components which will not significantly impair the biological properties of the peptides, for example the paratope binding capacity of antibodies (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing peptide aerosols without resort to undue experimentation.

Compositions suitable for oral administration may be presented as discrete units, in both immediate release or controlled release formulations, such as capsules, tablets, lozenges, each containing a predetermined amount of the active agent. Other compositions include suspensions in aqueous liquids or non-aqueous liquids such as a syrup, elixir or an emulsion.

Preparations for parenteral administration include sterile aqueous or non-aqueous solutions, suspensions, and emulsions as well as injectable drug delivery devices such as controlled release preparations. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

The agents may be combined, optionally, with a pharmaceutically-acceptable carrier. The term “pharmaceutically-acceptable carrier” as used herein means one or more compatible solid or liquid filler, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the agents of the present invention, and with each other, in a manner such that there is no interaction which would substantially impair the desired pharmaceutical efficacy.

When administered, the pharmaceutical preparations of the invention are applied in pharmaceutically-acceptable amounts and in pharmaceutically-acceptably compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Other delivery systems can include immediate release or controlled release formulations. Examples of controlled release formulations include time-release, delayed release or sustained release delivery systems. Such systems can reduce toxicity, increase efficacy and avoid repeated administrations of the platelet reducing agent, reducing peak-related side effects and increasing convenience to the subject and the physician. Many types of release delivery systems are available and known to those of ordinary skill in the art. They include but are not limited to polymer base systems such as poly(lactide-glycolide), copolyoxalates, polycaprolactones, lipids, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the platelet reducing agent is contained in a form within a matrix such as those described in U.S. Pat. Nos. 4,452,775, 4,675,189 and 5,736,152 and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant (or device) may be particularly suitable for treatment of subjects at elevated risk of particular disorders such as infection or cancer. Long-term release, as used herein, means that the implant is constructed and arranged to deliver levels of the active ingredient for at least 1 week, in some instances for at least 30 days, and in others for at least 60 days. In some aspects of the invention that involve longer-term treatment and prevention, it is desirable that the sustained release device release effective amounts of agent for at least 6 months, 1 year, 2 years or in some cases, 5 years or more. Long-term sustained release implants are well-known to those of ordinary skill in the art and include some of the release systems described above.

The agent of the invention should be administered for a length of time sufficient to provide either or both therapeutic and prophylactic benefit to the subject. Generally, the agent is administered for at least one day. In some instances, particularly where the subject is at risk of developing a disorder that benefits from heightened immune surveillance, the agent may be administered for the remainder of the subject's life. The rate at which the agent is administered may vary depending upon the needs of the subject and the mode of administration. For example, it may be necessary in some instances to administer higher and more frequent doses of the agent to a subject for example during or immediately following a an infection, for example. In still other embodiments, the same dose of agent may be administered throughout the treatment period which as described herein may extend throughout the lifetime of the subject. The frequency of administration may vary depending upon the characteristics of the subject. The agent may be administered daily, every 2 days, every 3 days, every 4 days, every 5 days, every week, every 10 days, every 2 weeks, every month, or more, or any time therebetween as if such time was explicitly recited herein.

In other aspects, the agents of the invention are administered with another agent, preferably an agent that would normally be indicated for the subject. In some embodiments, the agents may be administered substantially simultaneously with the other therapeutic agents. By substantially simultaneously, it is meant that the agent of the invention is administered to a subject close enough in time with the administration of the other therapeutic agent, whereby the two compounds may exert an additive or even synergistic effect.

The information generated according to the methods described above, in particular the information about expression levels of markers in a type of immune cell (“marker information”), can be included in a data structure (e.g., as part of a database), on a computer-readable medium, where the information may be correlated with other information pertaining to the markers or immune cells.

The invention will be more fully understood by reference to the following examples. These examples, however, are merely intended to illustrate the embodiments of the invention and are not to be construed to limit the scope of the invention.

### EXAMPLES

Introduction

DNA microarrays for identifying the mRNA and by extension protein constituents of living organisms while determining their time and space pattern of expression is an emerging technology (33). Microarrays are systematic arrays of cDNAs or oligonucleotides of known sequence that are printed or synthesized at discrete loci on a glass or silicon surface. Microarrays technology facilitates a more complete and inclusive experimental approach where alterations in the transcript level of entire genomes can be simultaneously assayed in response to a stimulus. This genome-wide approach to transcriptional analysis or “transcriptional profiling” provides comparative data on the relative expression level of individual transcripts within an organism, and relates this to alterations that occur as a consequence of a defined cellular stimulus. This new holistic approach has generated additional problems relating to data management and a requirement for sophisticated methods of analysis to extract biologically relevant data from the mass of primary information.

Oligonucleotide arrays, which were initially pioneered by Affymetrix, are generated using a combination of oligonucleotide synthesis and photolithography. A photolithographic mask is used to generate localized areas of photodeprotection on a glass slide that has been coated with linker molecules containing a photochemically removable protecting group. Specific dNTPs are then chemically coupled at the deprotected site facilitating the synthesis of specific oligonucleotide sequences. A series of different photolithographic masks are used with an intervening dNTP coupling reaction to generate the desired array. Other methods of generating oligonucleotide arrays rely on depositing a presynthesized oligonucleotide onto the array.

The first oligonucleotide arrays were generated using a combination of oligonucleotide synthesis and photolithography to synthesize specific oligonucleotides in a predetermined spatial orientation on a solid surface such as glass or silicon (34,35). Affymetrix (Santa Clara, Calif.), has pioneered this technology and currently has generated a number of different commercially available array products including human, mouse and various model organisms. The arrays are generated by attaching synthetic linker molecules that have been modified with a photochemically removable protecting group to a solid support such as glass or silicon. A photolithographic mask is then applied through which ultraviolet (UV) light is passed generating localized areas of photodeprotection to which protected dNTP are then attached in a chemical coupling reaction. Each photolithographic mask applied generates different areas of photodeprotection on the solid substrate and, using a combination of these masks with an intervening chemical coupling step, the desired probes are synthesized at the sites specified in the original design (36). An additional feature of oligonucleotide arrays is that each gene included on the array is represented by up to 20 different oligonucleotides spanning the entire length of the coding region of that gene. Moreover each of these oligonucleotides is paired with a second mismatch oligonucleotide in which the central base in the sequence has been changed. The combination of probe redundancy and inclusion of a mismatched control sequence greatly reduces the rate of false positives obtained from this type of approach. For expression profiling-based comparisons, fluorescently labelled probes are generated from test and reference samples. For Affymetrix-based oligonucleotide arrays, fluorescent probes are generated by reverse transcribing total RNA using an oligo-dT primer containing a T7 polymerase site. Amplification and labelling of the cDNA probe is achieved by carrying out an in vitro transcription reaction in the presence of a biotinylated dNTP, resulting in the linear amplification of the cDNA population (approximately 30-100-fold). The biotin-labelled cRNA probe generated from test and reference samples is then hybridised to separate oligonucleotide arrays, followed by binding to a streptavidin-conjugated fluorescent marker. Detection of bound probe is achieved following laser excitation of the fluorescent marker and scanning of the resultant emission spectra using a scanning confocal laser microscope. The differential fluorescent signal is then represented as alterations in transcriptional profile between the two samples compared.

Microarray technology is currently being used in a high-throughput approach to study gene expression and sequence variation on a genomic scale. The term expression profiling, however, encompasses a wide variety of different experimental strategies that use alterations in transcriptional profile as a means to explain the molecular basis of how specified experimental models respond to particular stimuli or changes in homeostasis, whether induced or occurring naturally. The questions being asked depend to a large extent on the design of the array experiment. In immunomodulation-profiling experiments the objective is to identify transcriptional changes that occur as a consequence of the transition from the resting to the activated phenotype. This approach also allows the identification of molecular fingerprints that facilitate the description of immunomodulatory activity of a given stimulus on a specific target immunocyte, thereby providing a molecular means to determine immunological impact of the chosen agent and definition of mechanism of action.

The application of microarray can be used to identify specific targets of defined genes that have clearly been implicated in immune stimulation. The objective of this approach is to define changes in transcriptional profile that occur in response to directed stimulation of relevant immunomodulatory receptors the resultant gene transcription modulation being the novel finding. The resulting altered expression profile can then be viewed as a blueprint by which that stimulus effects its cellular function and by extension system in vivo effects. In this approach the one has control over the question being asked as the experimental variables are directed and control controlled while being unique to a given system. Comparing transcriptional profiles as a means of defining downstream induced response has previously been validated by various researchers using alternative techniques such as differential display (37) and serial analysis of gene expression (38). The utilisation of microarray technology has, however, dramatically modified the experimental design required in this approach allowing the simultaneous identification of all potential targets. Currently the major drawback to using arrays in this manner is that it is entirely dependent on the state of knowledge of the genome under investigation.

Dendritic cells (DCs) are antigen-presenting cells that play a major role in initiating primary immune responses. We have utilized DNA microarrays to analyse the expression profile of human CD123+ dendritic cells or plasmacytoid precursor DC (ppDC) both in their unstimulated resting state and after CpG-DNA stimulation. Analysis of gene expression changes at the RNA level using oligonucleotide microarrays complementary to 12,560 human genes showed that ˜45.8% of the genes were expressed in DCs. The majority of these genes were not previously associated with DCs and included genes encoding secreted proteins, cell surface marker and receptors as well as genes involved in cell adhesion, signalling, and phagocytosis. Between 2 to 24 hour after CpG-DNA stimulation 335 to 582 genes were upmodulated and 314 to 740 down modulated respectively.

Isolating DCs From Peripheral Blood

Isolation of ppDC/pDC2 was performed as described previously (32). PBMC were isolated from citrate-stabilized buffy coats by centrifugation over Ficoll-Hypaque gradient. Briefly, 15 ml of the buffy coat was diluted 1/1 with PBS, underlayed with 15 ml of Ficoll-Hypaque solution, 1.077 g/l (Biochrom, Berlin, Germany), and centrifuged for 30 min at 1000×g. Cells at the interface were harvested and washed four times with HBSS. ppDC/DC2 were purified from PBMC based on CD123 and MHC class II expression by a combination of MACS separation and FACS sorting. For MACS separation. PBMCs were stained with PE-conjugated mAb against CD123 (PharMingen) and counterstained with anti-PE microbeads. CD123⁺ cells were positively selected on a column. For further purification, the enriched CD123⁺ cell fractions were sorted by FACS according to CD123⁺, HLA-DR⁺ expression. The purity was determined by flow cytometry and was >98%. The anti-CD123 mAb, clone 9F5, is a nonblocking Ab, so signalling by IL-3 via the receptor was possible after sorting.

Cells were cultivated in RPMI 1640 supplemented with 50 μM 2-ME, 2 mM L-glutamine, 100 U/ml penicillin G, 100 μg/ml streptomycin, 10 mM HEPES, and 10% FCS (Seromed, Berlin, Germany). IL-3 was added at a concentration of 500 U/ml.

The following CpG-oligodeoxynucleotide (ODN) was used in its phosphorothioate form: CpG-ODN 2006, 5′-TCGTCGTTTTGTCGTTTTGTCGTT-3′ (SEQ. ID NO:1) at 1.0 μM. Cultures were either mock stimulated or stimulated with CpG-ODN for the time periods 2 h, 8 h or 24 h as independent experiments. At the end of the stimulation time cells were pelleted by centrifugation, the supernatant removed, Rneasy lysis buffer was added and the cells were snap frozen at −70° C. until the RNA was extracted. Before RNA extraction lysates from multiple experiments were pooled in order to achieve 5 μg total RNA.

Preparation of cRNA and Gene Chip Hybridization

Total RNA was isolated using RNeasy isolation columns (Qiagen) and used to generate cRNA probes. Preparation of cRNA, hybridization, and scanning of the HuGeneFL arrays were performed according to the manufacturer's protocol (Affymetrix, Santa Clara, Calif.). Briefly, 5 μg of the RNA was converted into double-stranded cDNA by reverse transcription using a cDNA synthesis kit with an oligo(dT)₂₄ primer containing a T7 RNA polymerase promoter site added 3′ of the poly(T). After second-strand synthesis, labelled cRNA was generated from the cDNA sample by an in vitro transcription reaction supplemented with biotin-11-CTP and biotin-16-UTP. The labelled cRNA was purified by min in fragmentation buffer (40 mM Tris acetate, pH 8.1, 100 mM potassium acetate, 30 mM magnesium acetate) and then used to prepare 300 μl of hybridization mixture (100 mM MES, 0.1 mg/ml herring sperm DNA, (1 M sodium chloride, 10 mM Tris, pH 7.6, 0.005% Triton X-100) containing a mixture of control cRNAs for comparison of hybridization efficiency between arrays and for relative quantitation of measured transcript levels. Before hybridization, the cRNA samples were heated at 94° C. for 5 min, equilibrated at 45° C. for 5 min, and clarified by centrifugation (14,000×g) at room temperature for 5 min. Aliquots of each sample (10 μg of cRNA in 200 μl of the master mix) were hybridized to HuGeneFL Arrays at 45° C. for 16 h in a rotisserie oven set at 60 rpm then washed with non stringent wash buffer (6×saline/sodium phosphate/EDTA) at 25° C., followed by stringent wash buffer (100 mM MES (pH 6.7), 0.1 M NaCl, 0.01% Tween 20) at 50° C., stained with streptavidin-phycoerythrin (Molecular Probes), washed again with 6×saline/sodium phosphate/EDTA, stained with biotinylated anti-streptavidin IgG, followed by a second staining with streptavidin-phycoerythrin, and a third washing with 6×saline/sodium phosphate/EDTA. The arrays were scanned using the GeneArray scanner(Affymetrix). Data analysis was performed using GeneChip 4.0 software. The software includes algorithms that determine whether a gene is absent or present and whether the expression level of a gene in an experimental sample is significantly increased or decreased relative to a control sample. To assess differences in gene expression, we selected genes based on a sort score value equal or greater than 2. The sort score is calculated by Affymetrix software by using a combination of actual values of the average differences.

1. Banchereau, J., R. M. Steinman. 1998. Dendritic cells and the control of immunity. Nature 392:245.

2. Sallusto, F., M. Cella, C. Danieli, A. Lanzavecchia. 1995. Dendritic cells use macropinocytosis and the mannose receptor to concentrate macromolecules in the major histocompatibility complex class II compartment: downregulation by cytokines and bacterial products. J. Exp. Med. 182:389.

3. Koch, F., U. Stanzl, P. Jennewein, K. Janke, C. Heufler, E. Kampgen, N. Romani, G. Schuler. 1996. High level IL-12 production by murine dendritic cells: upregulation via MHC class II and CD40 molecules and downregulation by IL-4 and IL-10. J. Exp. Med. 184:741.

4. Flores-Romo, L., P. Bjorck, V. Duvert, C. van Kooten, S. Saeland, J. Banchereau. 1997. CD40 ligation on human cord blood CD34⁺ hematopoietic progenitors induces their proliferation and differentiation into functional dendritic cells. J. Exp. Med. 185:341.

5. Medzhitov, R., Jr C. A. Janeway. 1997. Innate immunity: the virtues of a nonclonal system of recognition. Cell 91:295.

6. Medzhitov, R., P. Preston-Hurlburt, Jr C. A. Janeway. 1997. A human homologue of the Drosophila Toll protein signals activation of adaptive immunity. Nature 388:394.

7. Aderem, A., R. J. Ulevitch. 2000. Toll-like receptors in the induction of the innate immune response. Nature 406:782.

8. Rissoan, M. C., V. Soumelis, N. Kadowaki, G. Grouard, F. Briere, M. de Waal, Y. J. Liu. 1999. Reciprocal control of T helper cell and dendritic cell differentiation. Science 283:1183.

9. Olweus, J., A. BitMansour, R. Warnke, P. A. Thompson, J. Carballido, L. J. Picker, F. Lund-Johansen. 1997. Dendritic cell ontogeny: a human dendritic cell lineage of myeloid origin. Proc. Natl. Acad. Sci. USA 94:12551.

10. Grouard, G., M. C. Rissoan, L. Filgueira, I. Durand, J. Banchereau, Y. J. Liu. 1997. The enigmatic plasmacytoid T cells develop into dendritic cells with interleukin (IL)-3 and CD40-ligand. J. Exp. Med. 185:1101.

11. Kohrgruber, N., N. Halanek, M. Groger, D. Winter, K. Rappersberger, M. Schmitt-Egenolf, G. Stingl, D. Maurer. 1999. Survival, maturation, and function of CD11c⁻ and CD11c⁺ peripheral blood dendritic cells are differentially regulated by cytokines. J. Immunol. 163:3250.

12. O'Doherty, U., M. Peng, S. Gezelter, W. J. Swiggard, M. Betjes, N. Bhardwaj, R. M. Steinman. 1994. Human blood contains two subsets of dendritic cells, one immunologically mature and the other immature. Immunology 82:487.

13. O'Doherty, U., R. M. Steinman, M. Peng, P. U. Cameron, S. Gezelter, I. Kopeloff, W. J. Swiggard, M. Pope, N. Bhardwaj. 1993. Dendritic cells freshly isolated from human blood express CD4 and mature into typical immunostimulatory dendritic cells after culture in monocyte-conditioned medium. J. Exp. Med. 178:1067.

14. Thomas, R., P. E. Lipsky. 1994. Human peripheral blood dendritic cell subsets: isolation and characterization of precursor and mature antigen-presenting cells. J. Immunol. 153:4016.

15. Thomas, R., L. S. Davis, P. E. Lipsky. 1993. Isolation and characterization of human peripheral blood dendritic cells. J. Immunol. 150:821.

16. Res, P. C., F. Couwenberg, F. A. Vyth-Dreese, H. Spits. 1999. Expression of pT mRNA in a committed dendritic cell precursor in the human thymus. Blood 94:2647.

17. Svensson, H., A. Johannisson, T. Nikkila, G. V. Alm, B. Cederblad. 1996. The cell surface phenotype of human natural interferon-α producing cells as determined by flow cytometry. Scand. J. Immunol. 44:164.

18. Siegal, F. P., N. Kadowaki, M. Shodell, P. A. Fitzgerald-Bocarsly, K. Shah, S. Ho, S. Antonenko, Y. J. Liu. 1999. The nature of the principal type 1. interferon-producing cells in human blood. Science 284:1835.

19. Svensson, H., B. Cederblad, M. Lindahl, G. Alm. 1996. Stimulation of natural interferon-α/β-producing cells by Staphylococcus aureus. J. Interferon Cytokine Res. 16:7.

20. Medzhitov, R., P. Preston-Hurlburt, E. Kopp, A. Stadlen, C. Chen, S. Ghosh, C. A. J. Janeway. 1998. MyD88. is an adaptor protein in the hToll/IL-1 receptor family signalling pathways. Mol. Cell 2:253.

21. Muzio, M., G. Natoli, S. Saccani, M. Levrero, A. Mantovani. 1998. The human toll signalling pathway: divergence of nuclear factor NFκB and JNK/SAPK activation upstream of tumor necrosis factor receptor-associated factor 6. (TRAF6). J. Exp. Med. 187:2097.

22. Kawai, T., O. Adachi, T. Ogawa, K. Takeda, S. Akira. 1999. Unresponsiveness of MyD88-deficient mice to endotoxin. Immunity 11:115.

23. Hacker, H., H. Mischak, T. Miethke, S. Liptay, R. Schmid, T. Sparwasser, K. Heeg, G. B. Lipford, H. Wagner. 1998. CpG-DNA-specific activation of antigen-presenting cells requires stress kinase activity and is preceded by non-specific endocytosis and endosomal maturation. EMBO J. 17:6230.

24. Hacker, H., H. Mischak, G. Hacker, S. Eser, N. Prenzel, A. Ullrich, H. Wagner. 1999. Cell type-specific activation of mitogen-activated protein kinases by CpG-DNA controls interleukin-12 release from antigen-presenting cells. EMBO J. 18:6973.

25. Hacker, H., R. M. Vabulas, O. Takeuchi, K. Hoshino, S. Akira, H. Wagner. 2000. Immune cell activation by bacterial CpG-DNA through myeloid differentiation marker 88 and tumor necrosis factor receptor-associated factor (TRAF) 6. J. Exp. Med. 192:595.

26. Hemmi, H., O. Takeuchi, T. Kawai, T. Kaisho, S. Sato, K. Hoshino, H. Wagner, K. Takeda, S. Akira. 2000. A novel Toll-like receptor that recognizes bacterial DNA. Nature 408:740.

27. Ishida, T., S. Mizushima, S. Azuma, N. Kobayashi, T. Tojo, K. Suzuki, S. Aizawa, T. Watanabe, G. Mosialos, E. Kieff, et al 1996. Identification of TRAF6, a novel tumor necrosis factor receptor-associated factor protein that mediates signalling from an amino-terminal domain of the CD40 cytoplasmic region. J. Biol. Chem. 271:28745.

28. Chu, W. M., D. Ostertag, Z. W. Li, L. Chang, Y. Chen, Y. Hu, B. Williams, J. Perrault, M. Karin. 1999. JNK2 and IKKβ are required for activating the innate response to viral infection. Immunity 11:721.

29. Sparwasser, T., E. S. Koch, R. M. Vabulas, K. Heeg, G. B. Lipford, J. Ellwart, H. Wagner. 1998. Bacterial DNA and immunostimulatory CpG oligonucleotides trigger maturation and activation of murine dendritic cells. Eur. J. Immunol. 28:2045.

30. Jakob, T., P. S. Walker, A. M. Krieg, M. C. Udey, J. C. Vogel. 1998. Activation of cutaneous dendritic cells by CpG-containing oligodeoxynucleotides: a role for dendritic cells in the augmentation of Th1 responses by immunostimulatory DNA. J. Immunol. 161:3042.

31. Hartmann, G., G. J. Weiner, A. M. Krieg. 1999. CpG DNA: a potent signal for growth, activation, and maturation of human dendritic cells. Proc. Natl. Acad. Sci. USA 96:9305.

32. Bauer, M., Redecke, V., Ellwart, J. W., Scherer, B., Krämer, J., Wagner, H., Lipford, G. B. 2001. Bacterial CpG-DNA triggers activation and maturation of CD11⁻, CD123⁺ dendritic cells. J. Immunol. 166:5000-5007.

33. Lockhart, D. J., and Winzeler, E. A. 2000. Nature 405, 827-836.

34. Lockhart D J, Dong H, Byrne M C et al. 1996. Expression monitoring by hybridization to high density oligonucleotide arrays. Nat Biotechnol 14:1675-1680.

35. Wodicka L, Dong H, Mittmann M et al. 1997. Genome-wide expression monitoring in Saccharomyces cerevisiae. Nat Biotechnol 15:1359-1367.

36. McGall G, Labadie J, Brock P et al. 1996. Light-directed synthesis of high-density oligonucleotide arrays using semiconductor photoresists. Proc Natl Acad Sci USA 93:13555-13560.

37. Tanaka H, Arakawa H, Yamaguchi T et al. 2000. A ribonucleotide reductase gene involved in a p53-dependent cell-cycle checkpoint for DNA damage. Nature 404:42-49.

38. Yu J, Zhang L, Hwang P M et al. 1999. Identification and classification of p53-regulated genes. Proc Natl Acad Sci USA 96:14517-22.

### Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

What is claimed is:

1. A method for identifying a plasmacytoid dendritic cell comprising
determining the level of expression of a PDC-specific set of markers in a test cell, and
comparing the level of expression with a control,
wherein a level of expression that is approximately identical to the control indicates that the test cell is a dendritic cell.

2. The method of claim 1, wherein the PDC-specific set of markers is a set of markers expressed in an unstimulated plasmacytoid dendritic cell.

3. The method of claim 1, wherein the level of expression is a level of mRNA expression.

4. The method of claim 3, wherein the level of mRNA expression is determined by Northern analysis, RT-PCR, or chip analysis.

5. The method of claim 1, wherein the level of expression is a level of protein expression.

6. The method of claim 1, wherein the level of protein expression is determined by FACS analysis.

7. The method of claim 1, wherein the PDC-specific set of markers comprises at least one marker, at least two markers, at least three markers, at least four markers, at least five markers, at least ten markers, at least twenty markers, or at least thirty markers.

8. The method of claim 1, wherein the PDC-specific set of markers comprises at least one marker expressed by natural killer (NK) cells.

9. The method of claim 8, wherein the at least one marker expressed by natural killer (NK) cells is selected from the group consisting of NKp30, ILT2, ILT3, ILT7, LAIR1, and NK4.

10. The method of claim 1, wherein the PDC-specific set of markers comprises a stimulatory molecule.

11. The method of claim 10, wherein the stimulatory molecule is selected from the group consisting of OX40 and 4-1BB ligand.

12. The method of claim 1, wherein PDC-specific set of markers comprises an integrin.

13. The method of claim 12, wherein the integrin is selected from the group consisting of β7 integrins, α7 integrins, α4 integrins, β2 integrins, β3 integrins and α3 integrins (CD49).

14. The method of claim 1, wherein the PDC-specific set of markers comprises a cell adhesion molecule.

15. The method of claim 14, wherein the cell adhesion molecule is selected from the group consisting of integrins, PECAM (CD31), ICAM-1 (CD54), ICAM-2 (CD102), ICAM-3 (CD50), siaoloadhesin (CD33), sialomucin (CD164), CD44, mucin (CD99) and MUC-1 (CD227).

16. The method of claim 1, wherein the PDC-specific set of markers comprises a cytokine receptor.

17. The method of claim 16, wherein the cytokine receptor is selected from the group consisting of IL-10 receptor, IL-1 receptor, TGF-β receptor, IL-6 receptor, IL-18 receptor and IL-17 receptor.

18. A method for a modulating plasmacytoid dendritic cell activity comprising
administering to a plasmacytoid dendritic cell an immunomodulatory agent having a receptor on the surface of the plasinacytoid dendritic cell in an amount effective to modulate plasmacytoid dendritic cell activity, following exposure of the plasmacytoid dendritic cell to an immunostimulatory nucleic acid,
wherein the receptor on the surface of the plasmacytoid dendritic cell is a PDC-specific marker.

19. The method of claim 18, wherein the immunomodulatory agent is an immunoinhibitory agent.

20. The method of claim 18, wherein the immunomodulatory agent is an immunostimulatory agent.

21. The method of claim 18, further comprising modulating an immune response that is therapeutically induced by administration of an immunostimulatory nucleic acid.

22. The method of claim 18, further comprising modulating an immune response selected from the group consisting of a response to a microbial infection, and an autoimmune disorder.

23. The method of claim 1, wherein the immunomodulatory agent is at least two, at three, at least four, or at least five immunomodulatory agents.

24. The method of claim 1, wherein the receptor on the surface of the plasmacytoid dendritic cell is a complement factor.

25. The method of claim 24, wherein the complement factor is selected from the group consisting of CD55 and CD46.

26. The method of claim 1, wherein the receptor on the surface of the plasmacytoid dendritic cell is a cell adhesion molecule.

27. The method of claim 26, wherein the cell adhesion molecule is an integrin, a mucin, a selectin, or a CAM.

28. The method of claim 26, wherein the cell adhesion molecules is selected from the group consisting of L-selectin (LECAM), CD164, CD44, CD43, CD87, CD47, CD81, CD162, CD147, CD11a, CD18, CD166 and CD49.

29. The method of claim 1, wherein the receptor on the surface of the plasmacytoid dendritic cell is a cell signaling receptor.

30. The method of claim 1, wherein the receptor on the surface of the plasmacytoid dendritic cell is a tyrosine kinase receptor.

31. The method of claim 1, wherein the receptor on the surface of the plasmacytoid dendritic cell is a phosphatase.

32. The method of claim 31, wherein the phosphatase is CD45.

33. The method of claim 1, wherein the receptor on the surface of the plasmacytoid dendritic cell is a growth factor receptor selected from the group consisting of a cytokine receptor and a chemokine receptor.

34. The method of claim 33, wherein the cytokine receptor is selected from the group consisting of IL-7 receptor (CD127), TNF receptor (CD120b), IL-4 receptor, CD132, IFN-γ receptor, IL-10 receptor, IL-1 receptor, TGFβ receptor, IL-6 receptor, IL-18 receptor, IL-17 receptor, IL-13 receptor, IL-15 receptor and IL-2 receptor.

35. The method of claim 33, wherein the chemokine receptor is selected from the group consisting of CD184 (CXCR4).

36. The method of claim 1, wherein the receptor on the surface of the plasmacytoid dendritic cell is an apoptosis modulating agent.

37. The method of claim 36, wherein the apoptosis modulating agent is CD95 and CD178.

38. The method of claim 1, wherein the receptor on the surface of the plasmacytoid dendritic cell is induced following CpG immunostimulation.

39. The method of claim 1, wherein the receptor on the surface of the plasmacytoid dendritic cell is up-regulated following CpG immunostimulation.

40. The method of claim 38 or 39, wherein the CpG immunostimulation is a 2 hour CpG immunostimulation or an 8 hour CpG immunostimulation.

41. The method of claim 1, wherein the receptor on the surface of the plasmacytoid dendritic cell is expressed in an unstimulated plasmacytoid dendritic cell.

42. The method of claim 1, wherein the immunomodulatory agent is selected from the group consisting of an antibody or antibody fragment specific for the receptor and a ligand for the receptor.

43. The method of claim 1, wherein the receptor on the surface of the plasmacytoid dendritic cell is ILT7, 4-1BB ligand, or OX-40.

44. A method of isolating plasmacytoid dendritic cells comprising
isolating from a bodily sample cells that express at least five PDC-specific markers, and
removing from the bodily sample cells that express a marker that is not a PDC-specific marker.

45. The method of claim 44, wherein the plasmacytoid dendritic cell is in a resting state.

46. The method of claim 44, wherein the plasmacytoid dendritic cell has been exposed to a CpG immunostimulatory nucleic acid.

47. The method of claim 44, wherein the PDC-specific markers comprise cell surface markers having a rank of greater than 10, greater than 25, greater than 50, greater than 75, greater than 100, greater than 125, or greater than 150 in Table 1a.

48. The method of claim 44, wherein the PDC-specific markers comprise receptors having a rank of greater than 7, greater than 10, or greater than 15 in Table 1b.

49. The method of claim 44, wherein the PDC-specific markers comprise at least one tyrosine kinase.

50. The method of claim 44, wherein the PDC-specific markers comprise at least one phosphatase.

51. The method of claim 44, wherein the PDC-specific markers comprise at least one apoptosis regulating molecule.

52. The method of claim 44, wherein the PDC-specific markers comprise at least one NK cell marker.

53. The method of claim 44, wherein the PDC-specific markers comprise at least one co-stimulatory molecule selected from the group consisting of OX-40 and 4-1BB ligand.

54. The method of claim 44, wherein the bodily sample is selected from the group consisting of peripheral blood, bone marrow or lymph node tissue.

55. A method for identifying a cell as a plasmacytoid dendritic cell comprising
obtaining a hybridization pattern by hybridizing a nucleic acid sample from a cell to an array of oligonucleotides at known locations on a substrate, and
comparing the hybridization pattern of the nucleic acid sample to a plasmacytoid expression database,
wherein the oligonucleotides are complementary to nucleic acid sequences from a plasmacytoid expression database, and
wherein a hybridization pattern of the nucleic acid sample that is approximately identical to the plasmacytoid expression database indicates that the cell is a plasmacytoid dendritic cell.

56. The method of claim 55, wherein the nucleic acid sequences from a plasmacytoid expression database are selected from the group consisting of nucleic acid sequences from Tables 1a and 1b.

57. The method of claim 55, wherein the nucleic acid sequences from a plasmacytoid expression database are selected from the group of nucleic acid sequences from Tables 1a and 1b that are cell surface markers, signaling markers and adhesion markers.

58. The method of claim 55, wherein the nucleic acid sample from the cell is amplified.

59. The method of claim 55, wherein the nucleic acid sequences from a plasmacytoid expression database have a known function.

60. The method of claim 55, wherein the plasmacytoid expression database is selected from the data of Tables 1a and 1b.

61. A method for identifying a subject responsive to treatment comprising
determining the level of expression of at least 5 PDC-specific markers in a plasmacytoid dendritic cell population harvested from a subject and exposed to a CpG immunostimulatory nucleic acid, and
comparing the level of expression of the at least 5 markers in the plasmacytoid dendritic cell population to a control,
wherein a level of expression that is approximately identical to the control indicates that the subject is responsive to treatment.

62. The method of claim 61, wherein the control is the data of PDC-specific markers in Tables 2a, 2b or 2c.

63. A method for evaluating a subject undergoing an immunomodulatory treatment comprising
determining an in vivo level of expression of a marker in the subject following administration of an immunomodulatory treatment, and
comparing the in vivo level of expression of the marker with a control,
wherein an in vivo level of expression of the marker that is approximately identical to the control is indicative of a response to the treatment in vivo.

64. The method of claim 63, wherein the control is a plasmacytoid expression database generated from plasmacytoid dendritic cells exposed to CpG immunostimulatory nucleic acids.

65. The method of claim 63, wherein the control is data of Tables 2a, 2b or 2c.

66. The method of claim 63, further comprising administering a second treatment to the subject.

67. The method of claim 66, wherein the second treatment down-regulates an immune response in the subject.

68. The method of claim 66, wherein the second treatment comprises administration of IL-10 or an antibody or antibody fragment specific for IL-10.

69. The method of claim 66, wherein the second treatment up-regulates an immune response in the subject.

70. The method of claim 66, wherein the second treatment comprises administration of OX-40 ligand or 4-1BB.

71. A method for identifying an agent that modulates plasmacytoid dendritic cell activity comprising
contacting a plasmacytoid dendritic cell with an agent,
determining the level of expression of a PDC-specific marker, and
comparing the level of expression of the PDC-specific marker to a control,
wherein an agent that induces a level of expression of a PDC-specific marker that is approximately identical or greater than the control is an agent that modulates plasmacytoid dendritic cell activity.

72. The method of claim 71, wherein the control comprises data of Tables 2a, 2b and 2c.

73. The method of claim 71, wherein the PDC-specific marker is an activation marker.

74. The method of claim 73, wherein the activation marker is selected from the group consisting of OX-40 and 4-1BB ligand.

75. The method of claim 71, wherein the PDC-specific marker is a marker having a rank of greater than 20, greater than 50, greater than 100, or greater than 150 in Table 2a.

76. The method of claim 71, wherein the PDC-specific marker is a marker having a rank of greater than 20, greater than 50, greater than 100, or greater than 150 in Table 2b.

77. The method of claim 71, wherein the PDC-specific marker is a marker having a rank of greater than 20, greater than 50, greater than 100, or greater than 150 in Table 2c.

78. A method of treating a subject to potentiate an immune response induced by administration of an immunostimulatory nucleic acid molecule comprising
administering to a subject in need thereof an immunostimulatory agent having a receptor on the surface of a plasmacytoid dendritic cell in an amount effective to stimulate a plasmacytoid dendritic cell,
wherein the agent is a ligand of the receptor or an antibody or antibody fragment specific for the receptor, and
wherein the receptor on the surface of the plasmacytoid dendritic cell is a PDC-specific marker.

79. The method of claim 78, wherein the PDC-specific marker is induced following exposure to CpG immunostimulatory nucleic acids.

80. The method of claim 78, wherein the PDC-specific marker is induced following exposure to CpG immunostimulatory nucleic acids for 2 hours, 8 hours, or 24 hours.

81. The method of claim 78, wherein the agent is 4-1BB receptor or OX-40 ligand.

82. The method of claim 78, wherein the agent is a chemokine or cytokine selected from the group consisting of IL-18, IL-15, IL-6 and IL-2.
